(19) United States
(12) Patent Application Publication
(10) Pub. No.: US 2009/0105327 A1
(43) Pub. Date: Apr. 23, 2009

(54) Dibenzocycloheptane compounds and pharmaceuticals containing these compounds

(75) Inventors: Stefan Laufer, Tuebingen (DE); Wolfgang Albrecht, Ulm (DE); Cornelia Greim, Weilheim/Teck (DE); Hans-Guenter Striegel, Blaubeuren (DE); Karola Tollmann, Brechen (DE)

Correspondence Address: ROTHWELL, FIGG, ERNST & MANBECK, P.C., 1425 K STREET, N.W., SUITE 800, WASHINGTON, DC, 20005, US

(73) Assignee: Merckle GMBH, Ulm (DE)

(21) Appl. No.: 11/914,078

(22) PCT Filed: May 12, 2006
(86) PCT No.: PCT/EP06/04488

§ 371 Date: Aug. 7, 2008

Related U.S. Application Data
(60) Provisional application No. 60/679,967, filed on May 12, 2005.

(30) Foreign Application Priority Data
May 12, 2005 (DE) 10 2005 022 020.7

(51) Int. Cl. A61K 31/335; C07D 337/12; C07D 209/04; C07D 313/12; A61P 25/00; A61P 3/00; A61P 35/00; A61P 37/00; A61K 31/38; A61K 31/404

(52) U.S. Cl. 514/415; 549/12; 548/469; 549/354; 514/431; 514/450

(57) ABSTRACT

The present invention relates to compounds of the formula I

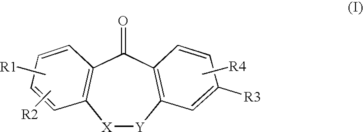

in which R1, R2, R3, R4, X and Y have the meanings indicated in the description. These compounds have immunomodulating effects, as well as an inhibiting or regulating effect on the release of IL-1β and/or TNF-α. They can therefore be used for the treatment of diseases associated with a disturbance of the immune system.

### CROSS-REFERENCE TO RELATED APPLICATION

This application is a National Phase of International Application No. PCT/EP2006/04488, filed May 12, 2006, which is a non-provisional of U.S. Provisional Application No. 60/679,967, filed May 12, 2005 and further claims priority from German Patent Application No. 10 2005 022 020.7, filed May 12, 2005.

### BACKGROUND OF THE INVENTION

1. Field of the Invention

The present invention relates to dibenzocycloheptane compounds of the formula I

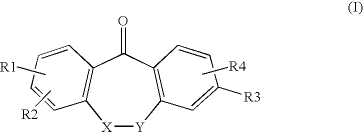

in which X, Y and R1 to R4 have the meanings indicated below, and to pharmaceutical compositions which comprise the compounds of the formula I. The compounds are inhibitors of interleukin-1β (IL-1β)- and tumor necrosis factor α (TNF-α) inhibitors, which can be used to treat inflammatory disorders.

2. Description of the Background Art

IL-1β and TNF-α protect from the body from infectious agents, tumors or tissue damage. However, in autoimmune diseases there is an increased production of IL-1β and TNF-α, possibly resulting in breakdown of bone and cartilage. Medicaments which regulate the release of IL-1β and TNF-α can therefore be used to treat inflammatory disorders.

A group of compounds which inhibit the release of IL-1β and TNF-α are disclosed in WO 98/32730. They correspond to the general formula

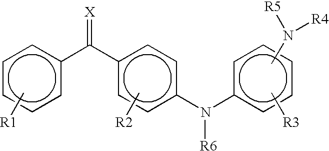

and can be used for the treatment and prophylaxis of asthma, allergies, rheumatoid arthritis, spondylarthritis, gout, atherosclerosis, chronic inflammatory bowel disease, proliferative and inflammatory skin disorders such as psoriasis and atopic dermatitis.

Further compounds of this group are described in WO 01/05744, WO 01/05745, WO 01/05746, WO 01/05749, WO 01/05751, WO 01/42189, WO 02/45752, WO 02/076447 and WO 03/018535 and in J. Med. Chem. 2003, 46, 5651-5662 and Bioorganic & Medicinal Chemistry Letters 14 (2004) 3601-3605. The effect of these compounds is, however, not entirely satisfactory.

### SUMMARY OF THE INVENTION

The present invention is therefore based on the object of providing compounds having an improved anti-inflammatory effect.

This object is achieved by the compounds of the formula I. The invention thus relates to dibenzocycloheptane compounds of the formula I

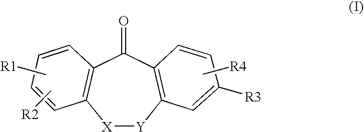

in which
one of the ring atoms X and Y is CH₂ and the other is O, S, SO, SO₂ or NR5;

### or —X—Y— is —CH₂—CH₂— or —CH═CH—;

R1 is H or C₁-C₆-alkyl;
R2 is H, halogen or C₁-C₄-alkyl-C≡C— which is optionally substituted by an amino group;
R3 is selected from:

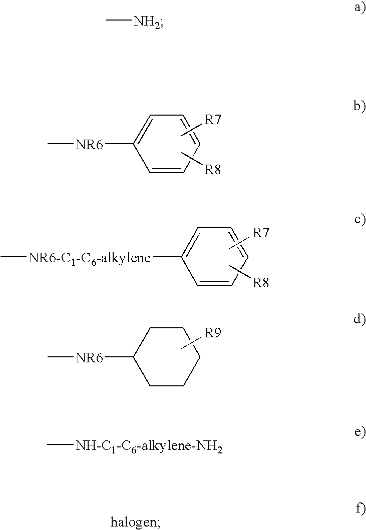

R4 is H, halogen or C₁-C₆-alkyl, or R3 and R4 are bonded to adjacent C atoms of the phenyl ring and form together with these C atoms a 5- or 6-membered aromatic or nonaromatic heterocycle having a nitrogen heteroatom, where the heterocycle may be substituted by one or two C₁-C₆-alkyl groups or may be fused to a cyclohexyl group;
R5 is H or C₁-C₆-alkyl;
R6 is H or C₁-C₆-alkyl;
R7 is selected from:

### H,

### NH₂,

mono-C₁-C₆-alkylamino,
di-C₁-C₆-alkylamino
C₁-C₆-alkyl-CONH—,
C₁-C₆-alkyl-NHCONH—,
C₁-C₆-alkyl-O—CO—NH—,
C₁-C₆-alkyl,
C₁-C₆-alkoxy,

### NO₂ or

halogen;
R8 is H, NH₂, mono-C₁-C₆-alkylamino, di-C₁-C₆-alkylamino, C₁-C₆-alkoxy or halogen;

### R9 is H or NH₂;

and the physiologically tolerated salts and the solvates of the compounds and of the salts thereof.

The term “alkyl” (also in connection with other groups such as haloalkyl, etc.) includes straight-chain and branched alkyl groups preferably having 1 to 6 or 1 to 4 carbon atoms, such as methyl, ethyl n- and i-propyl, n-, i- and t-butyl, sec-butyl, n-pentyl and n-hexyl.

The term “halogen” stands for a fluorine, chlorine, bromine or iodine atom, in particular for a fluorine or chlorine atom.

### DETAILED DESCRIPTION OF THE PREFERRED EMBODIMENT(S)

One embodiment of the invention are the compounds of the formula Ia:

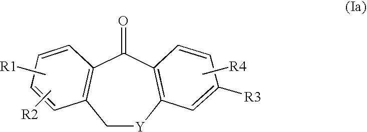

in which Y is O or S, and R1, R2, R3 and R4 have the abovementioned meanings.

A further embodiment are the compounds of the formula Iaa

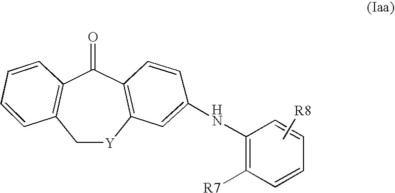

in which Y is O or S, and R7 and R8 have the abovementioned meanings. A further embodiment of the invention are the compounds of the formula Ib

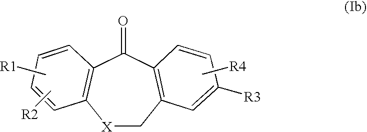

in which X is O or S, and R1, R2, R3 and R4 have the abovementioned meanings. A further embodiment are the compounds of the formula Ic

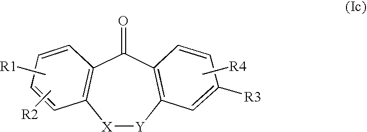

in which —X—Y— is —CH₂—CH₂— or —CH═CH—, and R1, R2, R3 and R4 have the abovementioned meanings.

A further embodiment are the compounds of the formula Ica:

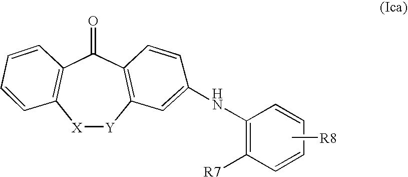

in which —X—Y— is —CH₂—CH₂— or —CH═CH—, and R7 and R8 have the above-mentioned meanings.

R1 and R2 are preferably H.

R3 is in one embodiment selected from the abovementioned formulae (b) to (e) and in particular (b) and (c). R3 is particularly preferably selected from

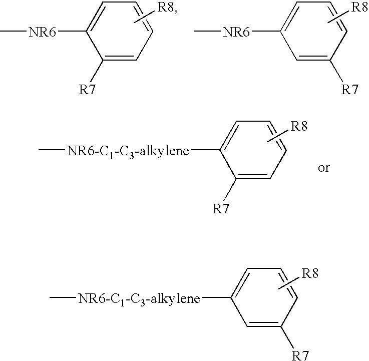

in which R7 and R8 have the abovementioned meanings.

R4, R5 and R6 are preferably H.

R7 is preferably NH₂, C₁-C₆-alkyl-CONH—, C₁-C₆-alkyl-NHCONH— or C₁-C₆-alkyl-O—CO—NH;

R8 is preferably H, NH₂ or halogen.

The invention also includes the physiologically tolerated salts of the compounds of the formula I. In the present case, they are in particular the acid addition salts. The acid addition salts are formed by employing inorganic acids such as hydrochloric acid, sulfuric acid or phosphoric acid or organic acids such as tartaric acid, citric acid, maleic acid, fumaric acid, malic acid, mandelic acid, ascorbic acid, gluconic acid, methanesulfonic acid, benzenesulfonic acid or toluenesulfonic acid and the like.

Where the compounds of the invention have centers of asymmetry, the invention likewise relates to the racemates and the individual optical isomers (enantiomers, diastereomers).

The invention also relates to the solvates of the compounds of the formula I or of the salts thereof, especially the hydrates.

The compounds of the invention are prepared by one of the processes illustrated below:

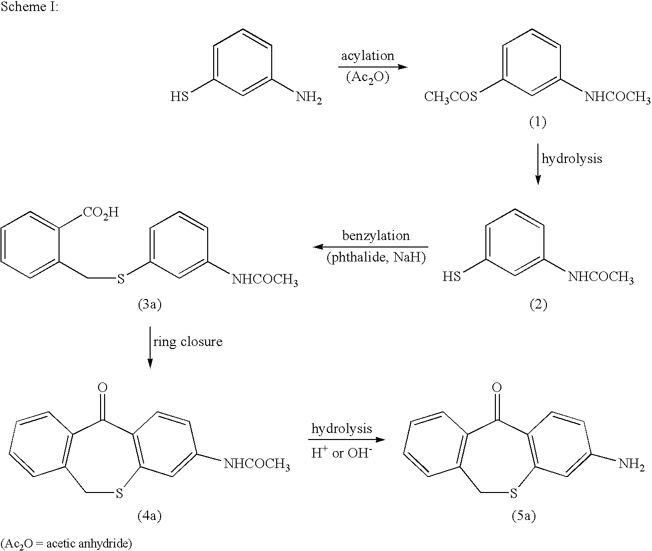

Scheme I illustrates by way of example the preparation of compounds of the formula I in which X is CH₂, and Y is S.

Starting from 3-aminothiophenol, the compound (1) acylated on the mercapto group and on the amino group is obtained by acylation, for example with acetic anhydride, in a conventional way. This compound is converted by partial hydrolysis with a base, for example an alkali metal hydroxide such as sodium hydroxide, into the compound (2). The compound (2) is reacted with phthalide, resulting in the compound (3a). Benzylation of the thiol group with phthalide takes place in the presence of a strong base such as sodium hydride, in a polar aprotic solvent such as dimethylformamide. The radicals R1 and R2 can be introduced by using the appropriately substituted phthalide. The preparation of halogen- or alkyl-substituted phthalides is known. If R2 is C₁-C₄-alkyl-C≡C—, this group can be introduced by the process described in WO 02/076447.

Compound (3a) can be converted by ring closure, for example with polyphosphoric acid in a polar organic solvent such as sulfolane, into the compound (4a). Acidic or alkaline hydrolysis affords compound (5a).

The oxepine derivatives, i.e. compounds of the formula I in which X is CH₂ and Y is O, can be prepared in an analogous manner starting from 3-aminophenol.

The compound of the formula (5a) is reacted further as shown in Scheme II.

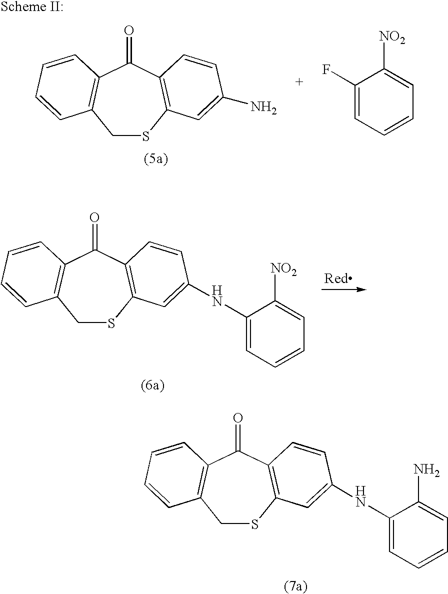

Scheme II illustrates the introduction of the radical R3 for the example of the preparation of the compound (7a). For this purpose, the compound (5a) is reacted with 2-nitrofluorobenzene in a nucleophilic aromatic substitution in a polar solvent to give the compound (6a). The nitro group in the compound (6a) is reduced in a conventional way, for example with Sn/HCl, to the amino group, resulting in the compound (7a).

Other radicals R3 can be introduced in an analogous manner. The corresponding oxepine compounds can also be obtained in an analogous manner.

An alternative method for introducing the radical R3 is illustrated in Scheme III.

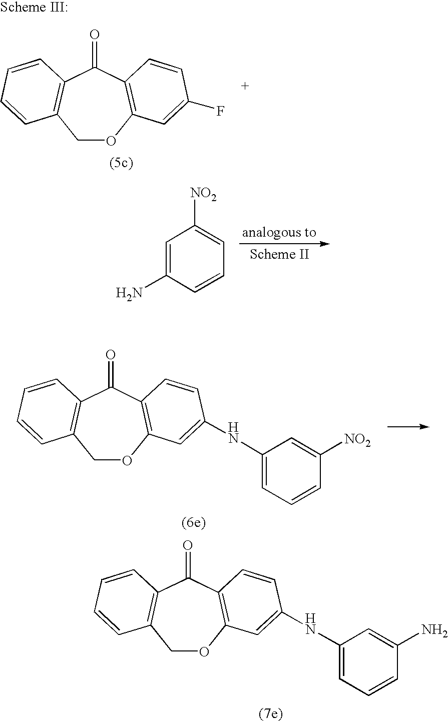

The first step in this method is also to introduce the radical R3 in a nucleophilic aromatic substitution by reacting the compound (5c) with 3-nitroaniline. The resulting compound (6e) is then converted into the compound (7e). The reactions are carried out as described above in connection with Scheme II. The introduction of other radicals R3 can take place in an analogous manner, as can the preparation of corresponding thiepine compounds.

Starting from a fluorinated compound (5) such as, for example, compound (5c), the radical R3 can also be introduced by reaction with a non-activated but reactive amine such as 2-aminobenzylamine, ethylenediamine or 1,2-diaminocyclohexane. This reaction is illustrated by way of example in Scheme IV.

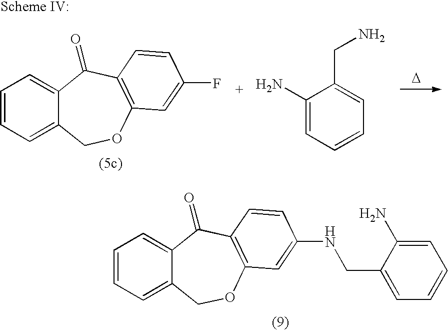

Reaction of the compound (5) with the appropriate amine expediently takes place without solvent at elevated temperature. A temperature in the range of about 80 to 150° C. is preferably used. The amine is generally employed in excess, especially in 5- to 25-fold excess.

Compounds of the formula I in which R3 is one of the radicals (b) or (c), and R7 is NH₂, can be converted into appropriate derivatives by modifying the amino group (R7═NH₂). Reactions of this type are summarized in Scheme V:

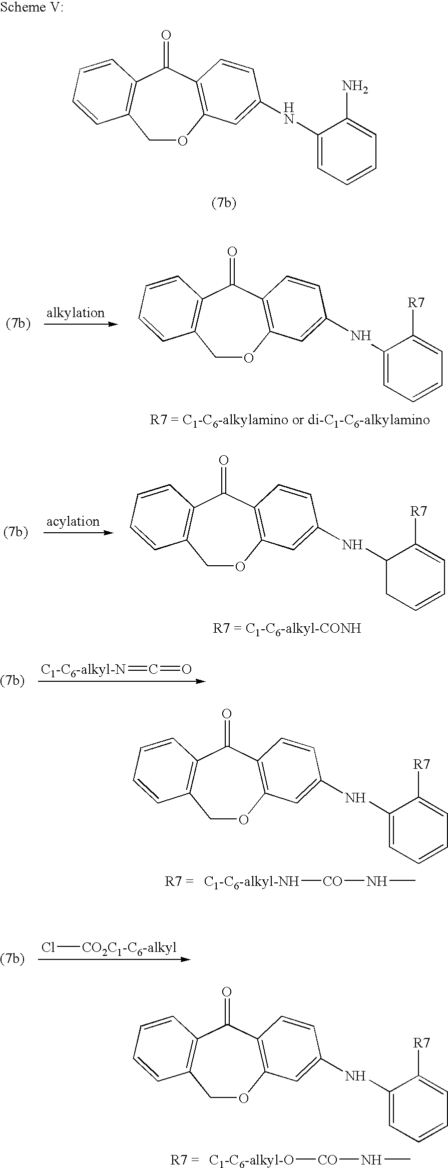

These involve conventional derivatizations of an amino group, and the reagents and reaction conditions required are well known to the skilled worker.

Compounds of the formula I in which R3 and R4 form, together with the carbon atom to which they are bonded, a 5- or 6-membered heterocycle having a nitrogen heteroatom can be prepared by reaction with a hydroxy keto compound. This reaction is illustrated in Scheme VI.

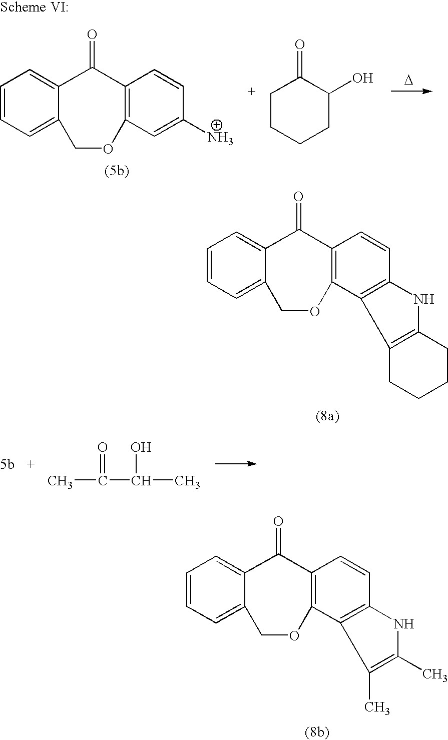

The reaction takes place in a polar organic solvent, for example an alkanol, such as methanol, ethanol or isopropanol and at elevated temperature. The reaction temperature is generally in the region of the boiling point of the reaction mixture.

Compounds of the formula I in which Y is SO or SO₂ are prepared by oxidizing the compounds in which Y is S in a conventional way, for example using per compounds such as m-chloroperbenzoic acid.

Compounds of the formula I in which X—Y is CH₂—CH₂ or CH═CH are prepared by the processes illustrated in Schemes VII to IX.

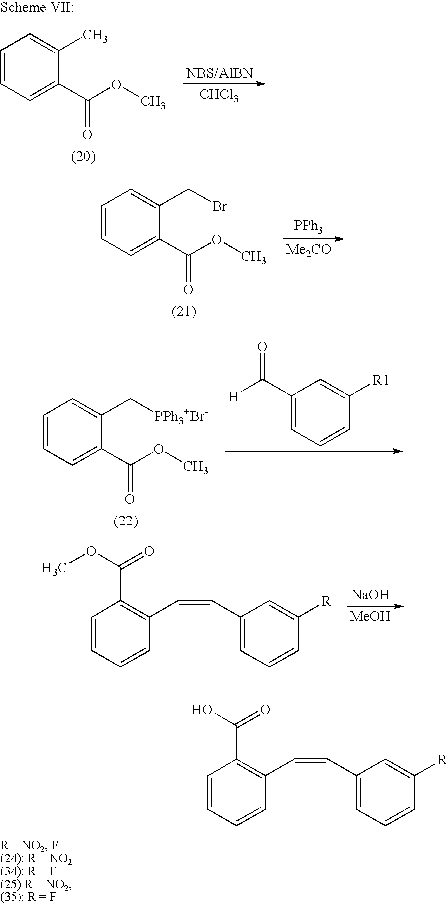

Scheme VII illustrates the preparation of the starting compounds for the example of compounds (25) and (35). (25) or (35) is prepared starting from methyl 2-methylbenzoate (20) in a conventional way by initially brominating (20), for example with N-bromosuccinimide (NBS) in an inert solvent such as methylene chloride or chloroform, in the presence of a free radical initiator such as azoisobutyronitrile. The resulting compound (21) is then reacted with triphenylphosphine to give the compound (22). This is converted in a Wittig reaction with 3-nitrobenzaldehyde or 3-fluorobenzaldehyde into the compound (24) or (34). Ester hydrolysis affords the compound (25) or (35).

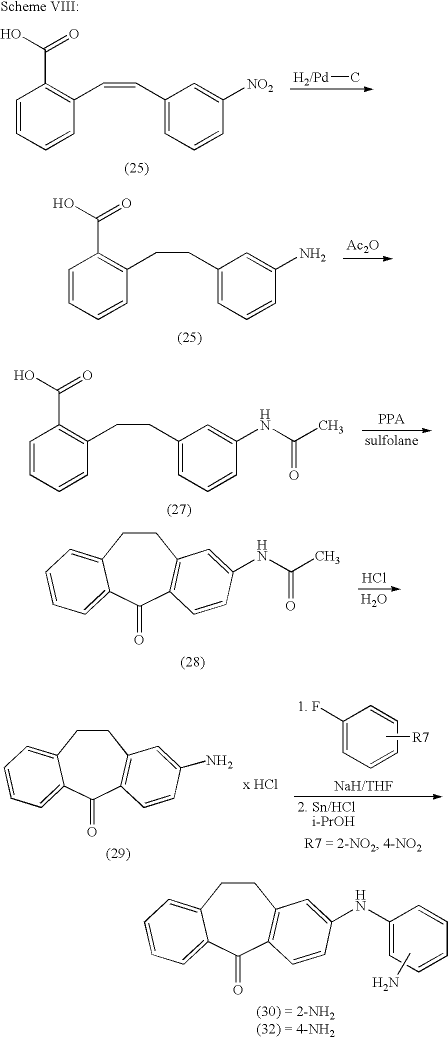

Scheme VII illustrates the preparation of the dibenzocycloheptanone compounds for the example of the compound (30) or (32). Starting from compound (25), initially the nitro group is reduced, for example with hydrogen/noble metal catalysts or tin/hydrochloric acid, resulting in the compound (26). The amino group in (26) is acylated in a conventional way, for example with acetic anhydride. Ring closure to give (28) then takes place using polyphosphoric acid (PPA) in an inert polar solvent such as sulfolane, at a temperature in the range from 100 to 200° C. The acetamido group then undergoes acidic hydrolysis, resulting in compound (29) which is converted by reaction with a nitro-substituted fluorobenzene in a nucleophilic aromatic substitution and subsequent reduction of the nitro group into the compound (30) or (32). These reactions are carried out as described in connection with Scheme I.

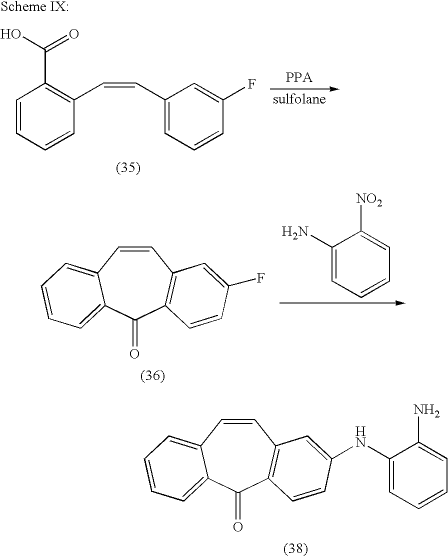

The compound (35) obtained as in Scheme VII is converted by ring closure with polyphosphoric acid in an inert polar solvent into compound (36). Compound (36) is reacted with 2-nitroaniline in a nucleophilic aromatic substitution and subsequent reduction of the nitro group to give the compound (38). These reactions take place in analogy to the reactions illustrated in Scheme II.

Starting from compound (36), it is possible to prepare other compounds of the formula I in analogy to the reactions illustrated in Scheme IV. Further compounds of the formula I can be prepared starting from compound (38) by modification of the amino group as shown in Scheme V.

Compounds of the formula I in which X is O, S, SO, SO₂ or NR5 are prepared by the known methods illustrated in Scheme X for the example of the oxepine compound. Further compounds can be prepared in analogy to Scheme V:

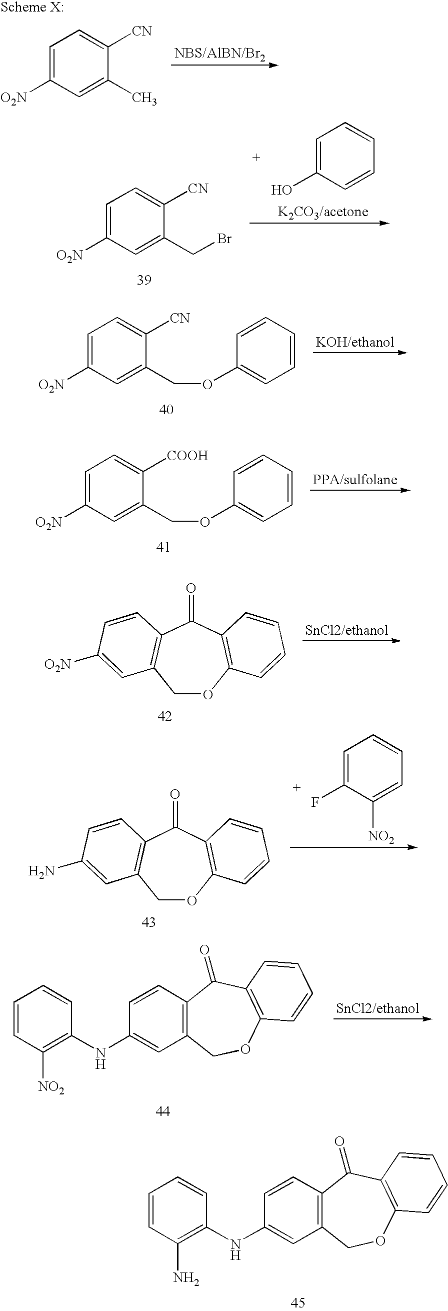

The compounds of the invention show in vitro and in vivo an immunomodulating effect and an effect inhibiting the release of TNF-α and IL-1β. The compounds of the invention are therefore suitable for the treatment of disorders associated with an impairment of the immune system. They are suitable for example for the treatment of autoimmune diseases, cancer, rheumatoid arthritis, gout, septic shock, osteoporosis, neuropathic pain, HIV dissemination, HIV dementia, viral myocarditis, insulin-dependent diabetes, periodontal disorders, restenosis, alopecia, T-cell depletion in HIV infections or AIDS, psoriasis, acute pancreatitis, rejection reactions with allogeneic transplants, allergy-related inflammation of the lungs, arteriosclerosis, multiple sclerosis, cachexia, Alzheimer's disease, stroke, icterus, ulcerative colitis, Crohn's disease, inflammatory bowel disease (IBD), ischemia, congestive heart failure, pulmonary fibrosis, hepatitis, glioblastoma, Guillain-Barré syndrome, systemic lupus erythematosus, adult respiratory distress syndrome (ARDS) and respiratory distress syndrome.

The compounds of the invention can be administered either as single therapeutic active ingredients or as mixtures with other therapeutic active ingredients. The compounds may be administered alone, but in general they are dosed and administered in the form of pharmaceutical compositions, i.e. as mixtures of the active ingredients with suitable pharmaceutical carriers or diluents. The compounds or compositions can be administered orally or parenterally, and they are preferably given in oral dosage forms.

The nature of the pharmaceutical composition or carrier or of the diluent depends on the desired administration form. Oral compositions may for example be in the form of tablets or capsules and may comprise conventional excipients such as binders (e.g. syrup, acacia, gelatin, sorbitol, tragacanth or polyvinylpyrrolidone), fillers (e.g. lactose, sugar, corn starch, calcium phosphate, sorbitol or glycine), lubricants (e.g. magnesium stearate, talc, polyethylene glycol or silicon dioxide), disintegrants (e.g. starch) or wetting agents (e.g. sodium lauryl sulfate). Liquid oral products may be in the form of aqueous or oily suspensions, solutions, emulsions, syrups, elixirs or sprays and the like. They may also be in the form of a dry powder which is prepared for reconstitution with water or another suitable carrier. Liquid products of this type may comprise conventional additives, for example suspending agents, flavorings, diluents or emulsifiers. Solutions or suspensions with conventional pharmaceutical carriers can be employed for parenteral administration.

The compounds or compositions of the invention can be administered to a mammal (human or animal) in a dose of about 0.5 mg to 100 mg per kg of body weight per day. They can be given in a single dose or in a plurality of doses. The range of effects of the compounds as inhibitors of TNF-α and IL-1β release has been investigated by means of the following test systems as described by Donat C. and Laufer S. in Arch. Pharm. Pharm. Med. Chem. 333, Suppl. 1, 1-40, 2000.

In Vitro Test Method with Human Whole Blood

Samples of human potassium EDTA whole blood (400 μl each) are mixed with the test substance and preincubated at 37° C. in a CO₂ incubator (5% CO₂; 95% moisture-saturated air) for 15 min. The samples are then stimulated with 1 μg/ml LPS (E. coli 026:B6) in a CO₂ incubator (5% CO₂; 95% moisture-saturated air) at 37° C. for 4 hours. The reaction is stopped by placing the samples on ice, adding DPBS buffer and subsequently centrifuging at 1000.g for 15 min. The amount of IL-1β and TNFα in the plasma supernatant is then measured by means of an ELISA.

In Vitro Test Method with PBMCs

1) Mononuclear cells (PBMCs) are isolated from 1:3-diluted human potassium EDTA whole blood by means of density gradient centrifugation (Histopaque®-1,077). These cells are washed twice with DPBS buffer, resuspended in macrophage SFM medium and adjusted to a cell count of 1*10⁶ cells/ml.

2) The resulting PBMCs suspension (samples each of 390 μl) is incubated with the test substance in a CO₂ incubator (5% CO₂; 95% moisture-saturated air) at 37° C. for 15 min. The samples are then stimulated with in each case 1 μg/ml LPS (E. coli 026:B6) in a CO₂ incubator (5% CO₂; 95% moisture-saturated air) at 37° C. for 4 hours. The reaction is stopped by placing the samples on ice, adding DPBS buffer and then centrifuging at 15 880*g for 12 min. The amount of IL-1β and TNFα in the plasma supernatant is then measured by means of an ELISA.

### Kinase Assay (p38 MAP Kinase Assay)

Microtiter plates were coated with 50 μl of ATF2 solution (20 μg/ml) at 37° C. for one hour. After washing with water three times, 50 μl of kinase mixture (50 mM tris-HCl 10 mM MgCl₂, 10 mM β-glycerol phosphate, 10 μg/ml BSA, 1 mM DTT, 100 μM ATP, 100 μM Na₃VO₄, 10 ng of activated p38α) with or without inhibitor were put into the wells and incubated at 37° C. for 1 hour. After washing three times, the plates were incubated with phosphorus-ATF-2 antibody at 37° C. for one hour. After washing again three times, a goat anti-rabbit IgG labeled with alkaline phosphatase was added at 37° C. for one hour (in order to retain the antibody phosphorylated protein-substrate complex). After washing three times, the alkaline phosphatase substrate solution (3 mM 4-NPP, 50 mM NaHCO₃, 50 mM MgCl₂, 100 μl/well) was added at 37° C. for 1.5 hours. The formation of 4-nitrophenolate was measured at 405 nm using a microtiter plate reader. The IC₅₀ values are calculated.

The results of the tests are shown in Table 1 below.

TABLE 1

| | | Inhibition of | |
|---|---|---|---|
| | p 38 MAP | TNF-α | IL-1β |
| Compound of | kinase assay | (whole blood) | (whole blood) |
| Example No. | IC₅₀ (μM) | IC₅₀ (μM) | IC₅₀ (μM) |
| 4 | 1.1 | — | — |
| 5 | 0.3 | — | — |
| 24 | 0.038 | — | — |
| 25 | 0.09 | 2.3 | 1.8 |
| 28 | 0.159 | — | — |

The following examples illustrate the invention without restricting it.

### Examples

The physicochemical data were established using the following materials and methods:

### 1. Melting Points:

Büchi Melting Point B-545 (thermodynamic correction)

### 2. NMR Spectroscopy:

### Bruker Advance 200 (200 MHz)

Internal standard: tetramethylsilane (TMS), δ [ppm]=0

### 3. IR Spectroscopy:

### Perkin Elmer Spectrum One (ATR Technique)

### 4. GC/MS:

### Hewlett Packard HP 6890 Series GC System

### Hewlett Packard HP 5973 Mass Selective Detector

### Method 1:

Inlet temperature: 250° C.

| Ramp rate | Final temperature | Hold time |
|---|---|---|
| [K/min] | [° C.] | [min] |
| | 100 | 1 |
| 10 | 160 | 10 |
| 15 | 200 | 15 |

### Method 2:

Inlet temperature: 250° C.

| Ramp rate | Final temperature | Hold time |
|---|---|---|
| [K/min] | [° C.] | [min] |
| | 100 | 1 |
| 10 | 160 | 10 |
| 15 | 200 | 15 |
| 10 | 270 | 20 |

### Method 3:

Inlet temperature: 250° C.

| Ramp rate | Final temperature | Hold time |
|---|---|---|
| [K/min] | [° C.] | [min] |
| | 160 | 1 |
| 10 | 240 | 5 |
| 10 | 270 | 15 |

### Method 4:

Inlet temperature: 250° C.

| Ramp rate | Final temperature | Hold time |
|---|---|---|
| [K/min] | [° C.] | [min] |
| | 160 | 1 |
| 10 | 240 | 5 |
| 10 | 270 | 30 |

### Method 5:

| Temperature [° C.] | Running time [min] |
|---|---|
| 100 | 0-1 |
| 100 → 160 | 1-7 |
| 160 | 7-17 |
| 160 → 200 | 17-19.67 |
| 200 | 34.67 |

### Method 6:

| Temperature [° C.] | Running time [min] |
|---|---|
| 150 | 0-1 |
| 150 → 230 | 1-9 |
| 230 | 9-14 |
| 230 → 270 | 14-22 |
| 270 | 22-40 |

1. General Method A¹ (the Footnotes Relate here and hereinafter to the List of References at the End of the Examples)

### Benzylation of the Thiophenol Compounds (Scheme I):

The reactant to be deprotonated is added in small portions to a suspension of sodium hydride in dimethylformamide. After gas evolution ceases, the second precursor is added and refluxed at about 160° C. The reaction mixture is cooled and, after addition of ice-water, acidified with hydrochloric acid (20%). The resulting precipitate is filtered off, washed with hydrochloric acid (10%) and dried over calcium chloride.

2. General Method B¹

### Ring Closure to Give Dibenzothiepine, Dibenzoxepine or Dibenzoazepine (cf., for Example, Scheme I)

The carboxylic acid compound to be subjected to the ring closure is dissolved in sulfolane under a protective gas atmosphere (argon) by heating to about 100° C. When the acid has completely dissolved, polyphosphoric acid is added, and the mixture is stirred at 10° C. for 2 h. Ice-water is then added, and the mixture is stirred at room temperature overnight. The precipitated product is filtered off and dried over calcium chloride.

3. General Method C²

### Reduction of the Nitrobenzene Compounds to Give the Corresponding Aniline Compounds (cf., for Example, Scheme II)

The compound to be reduced is dissolved in isopropanol by heating to reflux. When all the precursor has dissolved, concentrated hydrochloric acid is slowly added. Then tin is added in portions. After completion of the addition of tin, the mixture is refluxed for about 1.5-2 h. Cooling is followed by basification with sodium hydroxide solution (20%) and extraction with ethyl acetate (EtOAc). The combined ethyl acetate extracts are evaporated and purified by column chromatography.

4. General Method D⁷

Preparation of Stilbene Compounds (cf. Scheme VII)

The stilbenes (24) and (34) are synthesized by a Wittig reaction by stirring the stated amount of sodium methanolate solution (30% in MeOH) in methanol in a dry 500 ml three-neck flask at room temperature (RT) for 15 min and then adding the phosphonium salt to the mixture, stirring for a further 30 min and, after the mixture has been heated to 50° C., adding the appropriate aldehyde. Refluxing at 80° C. for the stated period is followed by stripping off the excess alcohol in vacuo. The residue is mixed with 150 ml of water and extracted with diethyl ether several times. Concentration of the combined organic phases after drying (Na₂SO₄) leads to the crude product, which is purified by column chromatography using CH₂Cl₂ or SiO₂.

5. General Method E⁸

Ester Hydrolysis (cf. Scheme VII)

The products obtained from the Wittig reaction are cleaved by dissolving the stated amount of ester in MeOH with heating, cautiously adding 20% strength sodium hydroxide solution, and refluxing at about 80° C. The alcohol is removed in vacuo, and the residue is extracted several times with CH₂Cl₂. Acidification of the aqueous phase with concentrated HCl results in the crude product in the form of a precipitate, which is filtered off. This precipitate is purified by digesting with diethyl ether, and the filtrate is concentrated to dryness in vacuo.

6. General Method F⁸

### Ring Closure to Give the Dibenzocycloheptanone Compounds (See Scheme VIII)

The ketones (28) and (36) are synthesized by dissolving the stated amount of carboxylic acid in sulfolane under an argon atmosphere in a dry 500 ml three-necked flask with heating, then adding polyphosphoric acid and refluxing at 110° C. Hydrolysis with ice-water is followed by stirring at RT, and the crude product which precipitates during this is filtered off and then purified by washing with H₂O.

7. General Method G¹²

Reduction of the Nitro Compounds Obtained by Substitution of the Dibenzocycloheptanone Compounds with 3-fluoronitrobenzene (cf. Scheme VIII)

The nitro compounds from the substitution reactions on compounds (29) and (36) are reduced by washing the filtration residue with isopropanol into a 100 ml round-bottomed flask and slowly adding conc. HCl while stirring at RT. After heating to 100° C., tin powder is added, followed by refluxing for 2 h. Cooling is followed by basification with 20% strength NaOH and repeated extraction with EtOAc; the combined org. phases are dried (Na₂SO₄) and then concentrated in vacuo.

General Method H⁴

### Reduction of Nitro Compounds

The nitro compound of Example 11 (7f) is reduced, for example, by dissolving the compound to be reduced in ethanol, adding tin(II) chloride dihydrate and stirring at 70° C. for about 2 h. After cooling to room temperature, ice-water is added to the mixture, which is basified with sodium hydroxide solution. The aqueous phase is extracted with ethyl acetate, saturated brine is used for washing, and the solvent is removed.

Preparation of Compounds of the Formula I in which Y═O or S

### Example 1

### 3-Amino-6,11-dihydrodibenzo[b,e]thiepin-11-one.HCl (5a)

### a) 2-(3-Acetamidophenylthiomethyl)benzoic Acid (3a)

### (1) 3-Acetylsulfanylacetanilide (1)³

This compound was prepared as described in Ref. (3).

### (2) 3-Acetamidothiophenol (2)¹

20.0 g (95.6 mmol) of 1,140 ml of sodium hydroxide solution (10%) and 60 ml of ethanol are stirred under reflux at 50° C. for about 20 min. The cooled mixture is then acidified with hydrochloric acid (20%). The product precipitates as a viscous mass and is extracted three times with diethyl ether. The ether is removed, resulting in the product.

GC (method 1) 12.0 min

MS m/z (%): 167 (52.5), 125 (100.0), 97 (19.9), 93 (13.2), 81 (30.2), 63 (10.5)

### (3) 2-(3-Acetamidophenylthiomethyl)benzoic Acid (3a)¹

By general method A, 4.05 g (92.8 mmol) of sodium hydride (55%), 15.0 g (89.7 mmol) of 2 and 12.15 g (90.6 mmol) of phthalide are employed with use of 90 ml of dimethylformamide. The reaction time is about 5 h. Working up takes place with about 180 ml of ice-water.

Yield: 23.7 g (87.6%); Melting point: 161-163° C.

¹H-NMR (DMSO-d₆) δ (ppm): 13.05 (s, 1H, —COOH), 9.95 (s, 1H, >NH), 7.85 (d, 1H, J=3.48 Hz, aryl H), 7.62 (s, 1H, aryl H), 7.48-7.30 (m, 4H, aryl H), 7.20 (t, 1H, J=7.87 Hz, aryl H), 6.97 (d, 1H, J=3.72 Hz, aryl H), 4.56 (s, 2H, —CH₂—S—), 2.03 (s, 3H, —CO—CH₃)

IR (ATR) (cm⁻¹): 1699, 1580, 1542, 1420, 1397, 1292, 1247, 1230, 778, 720.

### b) 3-Amino-6,11-dihydrodibenzo[b,e]thiepin-11-one.HCl (5a)

### (1) 3-Acetamido-6,11-dihydrodibenzo[b,e]thiepin-11-one (4a)¹

By general method B 10.0 g (33.2 mmol) of 3a, 50 ml of sulfolane and 100 ml (206 g) of polyphosphoric acid (PPA) are employed. A mixture of 3-acetamido-6,11-dihydrodibenzo[b,e]thiepin-11-one and 3-amino-6,11-dihydrodibenzo[b,e]thiepin-11-one is obtained but is not separated or purified.

¹H-NMR (DMSO-d₆) δ (ppm): 10.31 (s, 1H, >NH), 8.10 (d, 1H, J=4.40 Hz, aryl H), 7.80 (d, 1H, J=0.90 Hz, aryl H), 7.53-7.37 (m, 5H, aryl H), 4.22 (s, 2H, —CH₂—S—), 2.07 (s, 3H, —COCH₃)

IR (ATR) (cm⁻¹): 1690, 1618, 1584, 1568, 1507, 1371, 1267, 1244, 1065, 930

GC (method 2) 55.1 min

MS m/z (%): 283 (100.0), 250 (26.2), 241 (31.2), 213 (20.6), 208 (89.6), 197 (10.7), 184 (34.0), 180 (48.5), 152 (40.2), 139 (11.3), 89 (23.3), 63 (17.7).

### (2) 3-Amino-6,11-dihydrodibenzo[b,e]thiepin-11-one.HCl (5a)¹

The substance mixture (4a) from the previous stage is dissolved in about 120 ml of methanol by heating. Then 30 ml of concentrated hydrochloric acid are added, and the mixture is refluxed for about 2 h. It is then concentrated in vacuo. 10% hydrochloric acid is added to the residue, and the mixture is stirred and the product is allowed to crystallize. The filtered product is dried over calcium chloride.

Yield: 8.0 g (86.7% based on 4a); Melting point: 204-206° C.

¹H-NMR (DMSO-d₆) δ (ppm): 7.99 (d, 1H, J=4.26 Hz, aryl H), 7.51-7.42 (m, 2H, aryl H), 7.36-7.28 (m, 2H, aryl H), 6.56-6.51 (m, 2H, aryl H), 4.89 (s, 3H, —NH₃⁺), 4.11 (s, 2H, —CH₂—S—)

IR (ATR) (cm⁻¹): 1650, 1597, 1583, 1558, 1537, 1276, 1240, 931, 735, 685

GC (method 2) 40.2 min

MS m/z (%): 241 (94.7), 212 (62.4), 208 (100.0), 197 (10.5), 195 (10.3), 184 (15.3), 180 (72.0), 152 (32.4), 121 (10.3), 106 (15.5), 89 (25.5), 77 (10.4), 63 (21.1).

### Example 2

### 3-Amino-6,11-dihydrodibenzo[b,e]oxepin-11-one.HCl (5b)

### a) 2-(3-Acetamidophenoxymethyl)benzoic Acid (3b)¹

By general method A, 4.56 g (105 mmol) of sodium hydride (55%), 15.0 g (99.2 mmol) of 3-acetamidophenol and 13.5 g (101 mmol) of phthalide are employed with use of 90 ml of dimethylformamide. The reaction time is about 5 h. Working up takes place with about 180 ml of ice-water.

Yield: 14.2 g (50%); Melting point: 200-202° C.

¹H-NMR (DMSO-d₆) δ (ppm): 13.05 (s, 1H, —COOH), 9.93 (s, 1H, >NH), 7.94 (d, 1H, J=3.49 Hz, aryl H), 7.72-7.10 (m, 6H, aryl H), 6.66 (s, 1H, aryl H), 5.42 (s, 2H, —CH₂—O—), 2.02 (s, 3H, —CO—CH₃)

IR (ATR) (cm⁻¹): 1680, 1667, 1605, 1493, 1416, 1315, 1270, 1255, 1156, 1054, 1044, 732, 681.

### b) 3-Amino-6,11-dihydrodibenzo[b,e]oxepin-11-one.HCl (5b)

### (1) 3-Acetamido-6,11-dihydrodibenzo[b,e]oxepin-11-one (4b)

By general method B, 10.0 g (35.1 mmol) of 3b, 50 ml of sulfolane and 100 ml (206 g) of polyphosphoric acid are employed. A mixture of 3-acetamido-6,11-dihydrodibenzo[b,e]oxepin-11-one and 3-amino-6,11-dihydrodibenzo[b,e]oxepin-11-one, is obtained but is not separated or purified.

GC (method 2) 37.0 min

MS m/z (%): 267 (59.9), 225 (100.0), 196 (65.4), 180 (13.5), 168 (23.5), 152 (14.2), 139 (10.0), 115 (13.2), 89 (17.2), 77 (10.7), 63 (10.3).

### (2) 3-Amino-6,11-dihydrodibenzo[b,e]oxepin-11-one.HCl (5b)¹

The protective group is eliminated from the substance mixture 4b by the method described for 5a.

Yield: 5.0 g (54.5% based on 4b); Melting point: 210° C.

¹H-NMR (DMSO-d₆) δ (ppm): 7.89 (d, 1H, J=4.42 Hz, aryl H), 7.82-7.77 (m, 1H, aryl H), 7.63-7.44 (m, 3H, aryl H), 6.40 (dd, 1H, J₁=3.4 Hz, J₂=5.6 Hz, aryl H), 6.13 (d, 1H, J=1.07 Hz, aryl H), 5.15 (s, 2H, —CH₂—O—), 4.85 (s, 3H, —NH₃⁺)

IR (ATR) (cm⁻¹): 2922, 2852, 1642, 1622, 1598, 1542, 1459, 1301, 1252, 1151, 1124, 755, 698

GC (method 2) 35.0 min

MS m/z (%): 225 (100.0), 196 (61.9), 180 (15.0), 168 (18.3), 152 (9.8), 141 (9.0), 128 (4.4), 115 (10.5), 89 (15.5), 77 (6.1), 63 (10.4), 51 (8.4).

### Example 3

### 3-Fluoro-6,11-dihydrodibenzo[b,e]oxepin-11-one (5c)

### a) 2-(3-Fluorophenoxymethyl)benzoic Acid (3c)

By general method A, 2.30 g (52.7 mmol) of sodium hydride (55%), 5.61 g (50.0 mmol) of 3-fluorophenol and 6.80 g (50.7 mmol) of phthalide are employed with use of 50 ml of dimethylformamide. The reaction time is about 5 h. Working up takes place with about 90 ml of ice-water.

Yield: 4.37 g (48.5%); Melting point: 90-92° C.

¹H-NMR (CDCl₃) δ (ppm): 8.19-7.25 (m, 7H, aryl H), 6.80-6.67 (m, 1H, aryl H), 5.34 (s, 2H, —CH₂—O—)

IR (ATR) (cm⁻¹): 1690, 1613, 1595, 1581, 1490, 1311, 1287, 1273, 1165, 1139, 1042, 999, 957, 755, 735, 679, 671.

### b) 3-Fluoro-6,11-dihydrodibenzo[b,e]oxepin-11-one (5c)

By general method B, 5.00 g (20.3 mmol) of 3c, 25 ml of sulfolane and 48.5 ml (100 g) of polyphosphoric acid are employed. The mixture is worked up with about 150 ml of ice-water.

Yield: 2.30 g (49.7%); Melting point: 79-81° C.

¹H-NMR (CDCl₃) δ (ppm): 8.28 (dd, 1H, J₁=1.10 Hz, J₂=7.90 Hz, aryl H), 7.90 (dd, 1H, J₁=3.00 Hz, J₂=4.60 Hz, aryl H), 7.62-7.44 (m, 2H, aryl H), 7.37 (dd, 1H, J₁=2.95 Hz, J₂=4.15 Hz, aryl H), 6.90-6.71 (m, 2H, aryl H), 5.21 (s, 2H, —CH₂—O—)

¹³C-NMR (CDCl₃) δ (ppm): 189.52 (C¹¹), 165.96 (d, J=166.45 Hz, C³), 163.54 (d, J=32.68 Hz, C^{4a}), 140.24 (C^{6a}), 135.00 (C^{10a}), 134.38 (C⁸), 133.66 (d, J=47.00 Hz, C¹), 129.44 (C¹⁰), 129.31 (C⁹), 127.74 (C⁷), 122.17 (d, J=1.32 Hz, C^{11a}), 110.33 (d, J=10.90 Hz, C²), 106.86 (d, J=11.85 Hz, C⁴), 73.76 (C⁶)

IR (ATR) (cm⁻¹):1643, 1611, 1596, 1576, 1296, 1242, 1208, 1138, 1115, 1104, 1023, 851, 753, 695

GC (method 1) 18.8 min

MS m/z (%): 228 (100.0), 199 (73.6), 170 (24.9), 89 (13.6).

### Example 4

### 3-(2-Aminoanilino)-6,11-dihydrodibenzo[b,e]thiepin-11-one (7a)

### (1) 3-(2-Nitroanilino)-6,11-dihydrodibenzo[b,e]thiepin-11-one (6a)

By general method A, 0.45 g (10.3 mmol) of sodium hydride (55%), 1.00 g (3.60 mmol) of 5a and 0.51 g (3.60 mmol) of 2-fluoronitrobenzene are employed with use of 7.5 ml of dimethylformamide. The mixture is refluxed overnight (about 15 h). Working up takes place with about 50 ml of ice-water. The brownish red powder obtained in this way can be purified by column chromatography to give an orange-colored powder.

Yield: 570 mg (43.7%); Melting point: 186-188° C.

¹H-NMR (DMSO-d₆) δ (ppm): 9.28 (s, 1H, >NH), 8.17-8.07 (m, 2H, aryl H), 7.70-7.58 (m, 1H, aryl H), 7.58-7.48 (m, 3H, aryl H), 7.43-7.32 (m, 2H, aryl H), 7.72-7.07 (m, 3H, aryl H), 4.22 (s, 2H, —CH₂—S—)

IR (ATR) (cm⁻¹): 1590, 1578, 1498, 1441, 1347, 1251, 1233, 1167, 1146, 732.

### (2) 3-(2-Aminoanilino)-6,11-dihydrodibenzo[b,e]thiepin-11-one (7a)

By general method C, 0.50 g (1.38 mmol) of 6a is employed with use of 5 ml of isopropanol, 5 ml of concentrated hydrochloric acid and 1.2 g of tin.

Yield: 320 mg (43.4%); Melting point: 195° C.

IR (ATR) (cm⁻¹): 2922, 2853, 1615, 1583, 1564, 1497, 1479, 1457, 1275, 1238, 1136, 735

### Example 5

### 3-(2-Aminoanilino)-6,11-dihydrodibenzo[b,e]oxepin-11-one (7b)

### (1) 3-(2-Nitroanilino)-6,11-dihydrodibenzo[b,e]oxepin-11-one (6b)

By general method A, 0.52 g (12.0 mmol) of sodium hydride (55%), 1.00 g (3.82 mmol) of 5b and 0.54 g (3.82 mmol) of 2-fluoronitrobenzene are employed with use of 8 ml of dimethylformamide. The mixture is refluxed overnight (about 15 h). Working up takes place with about 50 ml of ice-water. The brownish red powder obtained in this way can be purified by column chromatography to give an orange-colored powder.

Alternatively, by general method A, 0.20 g (4.60 mmol) of sodium hydride (55%), 1.00 g (4.38 mmol) of 5c and 0.61 g (4.43 mmol) of 2-nitroaniline are employed with use of 5 ml of dimethylformamide. The mixture is refluxed overnight (about 15 h). Working up takes place with about 50 ml of ice-water.

Yield: 1.32 g (100%)

IR (ATR) (cm⁻¹): 1588, 1577, 1504, 1330, 1296, 1257, 1214, 1150, 1124, 735,713.

### (2) 3-(2-Aminoanilino)-6,11-dihydrodibenzo[b,e]oxepin-11-one (7b)

By general method C, 1.00 g (2.89 mmol) of 7b is employed with use of 15 ml of isopropanol, 15 ml of concentrated hydrochloric acid and 2.5 g of tin.

Alternatively, by general method H, 0.75 g (2.17 mmol) of 6b are dissolved in 4 ml of ethanol, 2.45 g (10.9 mmol) of tin(II) chloride dihydrate are added, and the mixture is stirred at 70° C. for about 2 h. After cooling to room temperature, ice-water is added to the mixture, which is basified with sodium hydroxide solution. The aqueous phase is extracted with ethyl acetate, saturated brine is used for washing, and the solution is concentrated.⁴

Yield: 135 mg (15%); Melting point: 122-124° C.

¹H-NMR (DMSO-d₆) δ (ppm): 8.16 (s, 1H, >NH), 7.96 (d, 1H, J=4.46 Hz, aryl H), 7.79 (d, 1H, J=3.65 Hz, aryl H), 7.60-7.46 (m, 3H, aryl H), 7.01-6.93 (m, 2H, aryl H), 6.78 (d, 1H, J=3.94 Hz, aryl H), 6.62-6.49 (m, 2H, aryl H), 6.08 (s, 1H, aryl H), 5.15 (s, 2H, —CH₂—O—), 4.86 (s, 2H, —NH₂)

¹³C-NMR (CDCl₃) δ (ppm): 188.70 (C¹¹), 163.45 (C^{4a}), 152.31 (C³), 142.26 (C^{2′}), 140.72 (C^{6a}), 135.54 (C^{10a}), 134.20 (C⁸), 132.10 (C¹), 129.57 (C¹⁰), 129.08 (C⁹), 127.56 (C⁷), 127.51 (C^{5′}), 127.01 (C^{4′}), 125.65 (C^{1′}), 119.45 (C^{6′}), 117.52 (C^{11a}), 116.67 (C^{3′}), 109.86 (C²), 102.25 (C⁴), 73.64 (C⁶)

IR (ATR) (cm⁻¹): 1587, 1559, 1498, 1459, 1297, 1276, 1253, 1229, 1154, 1118,746

GC (method 3) 29.4 min

MS m/z (%): 316 (100.0), 301 (15.5), 287 (14.2), 273 (12.5), 269 (13.7), 181 (24.1), 169 (22.7), 152 (24.2), 145 (17.7), 141 (10.2), 132 (14.3), 128 (13.6), 115 (18.3), 107 (11.2), 89 (29.6), 80 (22.7), 77 (16.7), 65 (29.3), 63 (16.9), 51 (11.6).

### Example 6

### 3-(4-Aminoanilino)-6,11-dihydrodibenzo[b,e]oxepin-11-one (7c)

### (1) 3-(4-Nitroanilino)-6,11-dihydrodibenzo[b,e]oxepin-11-one (6c)

By general method A, 0.52 g (12.0 mmol) of sodium hydride (55%), 1.00 g (3.82 mmol) of 5b and 0.54 g (3.82 mmol) of 4-fluoronitrobenzene are employed with use of 8 ml of dimethylformamide. The mixture is refluxed overnight (about 15 h). Working up takes place with about 50 ml of ice-water.

Yield: 1.32 g (100%)

IR (ATR) (cm⁻¹): 2854, 2923, 1585, 1572, 1500, 1322, 1293, 1250, 1109, 712

### (2) 3-(4-Aminoanilino)-6,11-dihydrodibenzo[b,e]oxepin-11-one (7c)

By general method C, 1.00 g (2.89 mmol) of 6c is employed with use of 15 ml of isopropanol, 15 ml of concentrated hydrochloric acid and 2.5 g of tin.

Yield: 186 mg (20.3%); Melting point: 131-133° C. (decomposition)

¹H-NMR (CDCl₃) δ (ppm): 8.15 (d, 1H, J=4.46 Hz, aryl H), 7.97-7.92 (m, 1H, aryl H), 7.51-7.43 (m, 2H, aryl H), 7.33-7.25 (m, 1H, aryl H), 7.03-7.97 (m, 2H, aryl H), 6.70-6.65 (m, 2H, aryl H), 6.52 (dd, 1H, J₁=3.32 Hz, J₂=5.60 Hz, aryl H), 6.33 (d, 1H, J=1.15 Hz, aryl H), 5.92 (s, 1H, >NH), 5.12 (s, 2H, —CH₂—O—), 3.56 (s, 2H, —NH₂) ¹³C-NMR (CDCl₃) δ (ppm): 188.29 (C¹¹), 163.37 (C^{4a}), 152.78 (C³), 143.85 (C^{4′}), 140.65 (C^{6a}), 135.52 (C^{10a}), 134.04 (C⁸), 131.86 (C¹), 130.56 (C^{1′}), 129.51 (C¹⁰), 128.92 (C⁹), 127.39 (C⁷), 125.41 (2C, C^{2′} and C^{6′}), 116.98 (C₁₁ₐ), 115.80 (2C, C^{3′} and C^{5′}), 109.62 (C²), 101.50 (C⁴), 73.52 (C⁶)

IR (ATR) (cm⁻¹): 1626, 1589, 1564, 1510, 1329, 1301, 1277, 1256, 1156, 1120, 826

GC (method 4) 32.5 min

MS m/z (%): 316 (100.0), 287 (10.9), 281 (10.5), 253 (6.1), 207 (51.3), 181 (7.0), 107 (8.2).

### Example 7

### 3-(2-Fluoro-4-aminoanilino)-6,11-dihydrodibenzo[b,e]oxepin-11-one (7d)

### (1) 3-(2-Fluoro-4-nitroanilino)-6,11-dihydrodibenzo[b,e]oxepin-11-one (6d)

By general method A, 0.52 g (12.0 mmol) of sodium hydride (55%), 1.00 g (3.82 mmol) of 5b and 0.54 g (3.82 mmol) of 3-fluoronitrobenzene are employed with use of 8 ml of dimethylformamide. The mixture is refluxed overnight (about 15 h). Working up takes place with about 50 ml of ice-water.

Yield: 0.80 g (57.5%);

IR (ATR) (cm⁻¹): 1588, 1575, 1528, 1505, 1489, 1320, 1290, 1271, 1251, 1188, 1154, 1122, 710,678

### (2) 3-(2-Fluoro-4-aminoanilino)-6,11-dihydrodibenzo[b,e]oxepin-11-one (7d)

By general method C, 0.80 g (2.20 mmol) of 6d is employed with use of 10 ml of isopropanol, 10 ml of concentrated hydrochloric acid, and 2.0 g of tin.

Yield: 150 mg (20.4%); Melting point: 123-125° C.

¹H-NMR (CDCl₃) δ (ppm): 8.16 (d, 1H, J=4.46 Hz, aryl H), 7.94 (d, 1H, J=3.57 Hz, aryl H), 7.51-7.43 (m, 2H, aryl H), 7.33-7.25 (m, 1H, aryl H), 7.10 (t, 2H, J=8.71 Hz, aryl H), 6.55-6.42 (m, 2H, aryl H), 6.30 (d, 1H, J=1.09 Hz, aryl H), 5.74 (s, 1H, >NH), 5.13 (s, 2H, —CH₂—O—), 3.77 (s, 2H, —NH₂)

¹³C-NMR (CDCl₃) δ (ppm): 188.51 (C¹¹), 163.29 (C^{4a}), 157.46 (d, J=121.73 Hz, C²), 152.39 (C³), 145.44 (d, J=5.10 Hz, C⁴), 140.60 (C^{6a}), 135.49 (C^{10a}), 133.99 (C⁸), 132.05 (C¹), 129.49 (C¹⁰), 128.95 (C⁹), 127.43 (C⁷), 127.07 (d, J=12.08 Hz, C⁶), 117.79 (d, J=7.35 Hz, C^{1′}), 117.37 (C^{11a}), 110.69 (d, J=5.95 Hz, C^{5′}), 109.66 (C²), 102.87 (d, J=11.60 Hz, C^{3′}), 101.85 (C⁴), 73.54 (C⁶)

IR (ATR) (cm⁻¹): 1624, 1589, 1564, 1517, 1494, 1299, 1277, 1229, 1155, 1120

GC (method 4) 30.5 min

MS m/z (%): 334 (100), 305 (10.5), 181 (7.8), 152 (8.7), 125 (11.2).

### Example 8

### 2,11,12,13,14-Pentahydro-10H-(benzo[e]oxepine)[2,3-c]carbazol-7-one (8a)

1.00 g (3.82 mmol) of 5b and 0.44 g (3.82 mmol) of 2-hydroxycyclohexanone are dissolved in ethanol and refluxed for about 16 h. After the reaction is complete, ice-water is added. The resulting precipitate is filtered off and purified. A pale yellow powder is obtained.

Yield: 1.12 g (96.6%); Melting point: 226-228° C.

¹H-NMR (DMSO-d₆) δ (ppm): 11.17 (s, 1H, >NH), 7.91-7.72 (m, 2H, aryl H), 7.68-7.44 (m, 2H, aryl H), 7.01 (d, 1H, J=4.38 Hz, aryl H), 5.77 (s, 1H, C⁹H), 5.31 (s, 2H, —CH₂—O), 2.85 (s, 2H, >C¹¹H₂), 2.66 (s, 2H, >C¹⁴H₂), 1.78 (s, 4H, —C¹²H₂—C¹³H₂) ¹³C-NMR (CDCl₃) δ (ppm): 190.75 (C⁷), 157.81 (C¹⁴c), 141.29 (C^{2a}), 140.16 (C^{9a}), 135.46 (C^{6a}), 133.33 (C^{10a}), 131.79 (C⁴), 129.39 (C⁶), 128.91 (C⁵), 127.30 (C³), 124.89 (C⁸), 118.19 (C^{14b}), 117.24 (C^{7a}), 112.42 (C^{14a}), 106.00 (C⁹), 74.04 (C²), 23.29 (C¹³), 23.13 (C¹²), 23.06 (C¹¹), 22.62 (C¹⁴)

IR (ATR) (cm⁻¹): 1607, 1586, 1568, 1349, 1314, 1282, 1236, 1164, 1098, 751, 736, 707

GC (method 2) 50.1 min

MS m/z (%): 303 (100), 286 (10.7), 274 (50.2), 258 (14.1), 246 (21.2), 207 (18.5).

### Example 9

### 2-Hydro-11,12-dimethyl-10H-(benzo[e]oxepine)[2,3-e]indol-7-one (8b)

1.00 g (3.82 mmol) of 5b and 0.34 g (3.82 mmol) of 3-hydroxybutan-2-one are dissolved in 15 ml of ethanol and refluxed overnight (about 16 h). Ice-water is added to the mixture, and the resulting precipitate is filtered off. A yellowish green powder is obtained by recrystallization from methanol.

Yield: 0.11 g (10.4%); Melting point: 160° C.

¹H-NMR (DMSO-d₆) δ (ppm): 11.18 (s, 1H, >NH), 7.95-7.75 (m, 2H, aryl H), 7.63-7.47 (m, 3H, aryl H), 6.99 (d, 1H, J=4.40 Hz, aryl H), 5.33 (s, 2H, —CH₂—O—), 2.32 (s, 3H, >C¹¹—CH₃), 2.26 (s, 3H, >C¹²—CH₃)

¹³C-NMR (CDCl₃) δ (ppm): 190.74 (C⁷), 158.09 (C^{12b}), 141.21 (C^{2a}), 139.84 (C^{9a}), 135.56 (C^{6a}), 131.81 (C⁴), 130.14 (C¹¹), 129.43 (C⁶), 128.90 (C⁵), 127.24 (C³), 124.78 (C⁸), 119.16 (C^{12a}), 117.25 (C^{7a}), 109.70 (C¹²), 105.83 (C⁹), 74.05 (C²), 11.17 (11-methyl), 10.90 (12-methyl)

IR (ATR) (cm⁻¹): 2922, 1592, 1568, 1341, 1307, 1268, 1249, 1164, 752,708

GC (method 2) 43.1 min

MS m/z (%): 277 (100), 262 (22), 248 (58.2), 234 (18.5), 232 (15.1), 124 (8.5).

### Example 10

### 3-(3-Aminoanilino)-6,11-dihydrodibenzo[b,e]oxepin-11-one (7e)

### (1) 3-(3-Nitroanilino)-6,11-dihydrodibenzo[b,e]oxepin-11-one (6e)

By general method A, 0.20 g (4.58 mmol) of sodium hydride (55%), 0.61 g (4.44 mmol) of 3-nitroaniline and 1.00 g (4.38 mmol) of 5c are employed with use of 5 ml of dimethylformamide. The mixture is refluxed overnight (about 15 h). Working up takes place with about 50 ml of ice-water.

Yield: 1.40 g (92.3%);

IR (ATR) (cm⁻¹): 2923, 2854, 1587, 1528, 1479, 1458, 1350, 1325, 1292, 1250, 1119, 1100, 712.

### (2) 3-(3-Aminoanilino)-6,11-dihydrodibenzo[b,e]oxepin-11-one (7e)

By general method C, 1.00 g (2.89 mmol) of 6e is employed with use of 15 ml of isopropanol, 15 ml of concentrated hydrochloric acid and 2.5 g of tin.

Yield: 71 mg (7.8%); Melting point: ˜150° C. (decomposition)

¹H-NMR (CDCl₃) δ (ppm): 8.19 (d, 1H, J=4.43 Hz, aryl H), 7.95 (dd, 1H, J₁=4.43 Hz, J₂=2.80 Hz, aryl H), 7.80-7.71 (m, 1H, aryl H), 7.53-7.46 (m, 2H, aryl H), 7.36-7.32 (m, 1H, aryl H), 7.18-7.06 (m, 1H, aryl H), 6.73-6.36 (m, 4H, aryl H), 6.09 (s, 2H, —NH₂), 5.16 (s, 2H, —CH₂—O—), 4.68 (s, 1H, >NH)

¹³C-NMR (CDCl₃) δ (ppm): 188.47 (C¹¹), 163.16 (C^{4a}), 150.51 (C³), 147.47 (C³), 141.05 (C^{1′}), 140.58 (C^{6a}), 135.48 (C^{10a}), 133.94 (C⁸), 132.01 (C¹), 130.23 (C¹⁰), 129.50 (C⁹), 128.99 (C^{5′}), 127.46 (C⁷), 117.84 (C^{11a}), 111.28 (C²), 110.86 (C^{6′}), 110.55 (C^{4′}), 107.31 (C^{2′}), 103.38 (C⁴), 73.54 (C⁶)

IR (ATR) (cm⁻¹): 1585, 1569, 1490, 1459, 1297, 1276, 1254, 1230, 1156, 1121, 759, 701

GC (method 4) 32.8 min

MS m/z (%): 316 (100.0), 287 (13.8), 281 (9.6), 223 (21.3), 207 (39.2), 181 (9.0), 106 (10.0).

### Example 11

### 3-(2,4-Diaminophenylamino)-6,11-dihydrodibenzo[b,e]oxepin-11-one (7f)

### (1) 3-(2,4-Dinitrophenylamino)-6,11-dihydrodibenzo[b,e]oxepin-11-one (6f)

By general method A, 0.10 g (2.30 mmol) of sodium hydride (55%), 0.40 g (2.21 mmol) of 2,4-dinitroaniline and 0.50 g (2.19 mmol) of 5c are employed with use of 5 ml of dimethylformamide. The mixture is refluxed overnight (about 15 h). Working up takes place with about 50 ml of ice-water and acidification with HCl. The precipitate which has been filtered off is purified by column chromatography on a silica gel column with dichloromethane as mobile phase. The yellow product is obtained in this way.

Yield: 720 mg (84.0%);

¹H-NMR (CDCl₃) δ (ppm): 10.01 (s, 1H, aryl H), 9.20 (d, 1H, J=0.99 Hz, aryl H), 8.46-8.27 (m, 2H, aryl H), 7.99-7.91 (m, 1H, aryl H), 7.64-7.28 (m, 3H, aryl H), 7.13-6.97 (m, 2H, aryl H), 5.26 (s, 2H, —CH₂—O—), >NH not detected

¹³C-NMR (CDCl₃) δ (ppm): 189.36 (C11), 162.33 (C4a), 144.77 (C3), 143.12 (C1′), 140.18 (C6a), 138.48 (C4′), 134.96 (C10a), 134.32 (C8), 132.95 (C1), 129.92 (C10), 129.50 (C9+C2′), 127.91 (C7), 123.83 (C5′), 123.41 (C2), 116.96 (C11a+C6′), 116.80 (C3′), 114.14 (C4), 73.73 (C6)

IR (ATR) (cm⁻¹): 1594, 1521, 1503, 1337, 1310, 1296, 1281, 1266, 1246, 1140, 1125.

### (2) 3-(2,4-Diaminophenylamino)-6,11-dihydrodibenzo[b,e]oxepin-11-one (7f)

By general method H, 0.72 g (1.84 mmol) of 6f is employed with use of 4 ml of ethanol and 4.00 g (18.4 mmol) of tin(II) chloride dihydrate. The resulting crude product is purified by MPLC on an RP18 silica gel column with acetonitrile/water 6+4 mobile phase.

Yield: 30 mg (4.9%);

¹H-NMR (CDCl₃) δ (ppm): 8.15 (d, 1H, J=4.40 Hz, aryl H), 7.94 (d, 1H, J=3.34 Hz, aryl H), 7.57-7.40 (m, 2H, aryl H), 7.36-7.23 (m, 1H, aryl H), 6.87 (d, 1H, J=3.85 Hz, aryl H), 6.44 (d, 1H, J=4.33 Hz, aryl H), 6.20-6.05 (m, 3H, aryl H), 5.50 (s, 1H, >NH), 5.12 (s, 2H, —CH₂—O—), 3.68 (s, 4H, —NH₂)]

¹³C-NMR (CDCl₃) δ (ppm): 188.50 (C11), 163.46 (C4a), 153.73 (C3), 146.51 (C4′), 144.53 (C2′), 140.66 (C6a), 135.46 (C10a), 134.10 (C8), 131.88 (C1), 129.50 (C10), 129.45 (C6′), 128.93 (C9), 127.41 (C7), 116.94 (C11a), 116.14 (C1′), 109.21 (C2), 106.12 (C5′), 101.93 (C4), 101.32 (C3′), 73.51 (C6)

IR (ATR) (cm⁻¹): 1622, 1590, 1563, 1514, 1468, 1384, 1300, 1276, 1255, 1235, 1155, 1119, 922, 758, 700.

### Example 12

### 3-(2-Aminobenzylamino)-6,11-dihydrodibenzo[b,e]oxepin-11-one (9)

2.27 g (18.6 mmol) of 2-aminobenzylamine are melted by heating. 0.25 g (1.10 mmol) of 5c is added in portions to the melt. After the addition is complete, the mixture is stirred for 4 h. Ice-water is added to the mixture, which is extracted with dichloromethane. The product obtained in this way is purified by column chromatography to give a yellowish orange powder.⁵

Yield: 160 mg (44.0%); Melting point: 72° C.

¹H-NMR (CDCl₃) δ (ppm): 8.17 (d, 1H, J=4.46 Hz, aryl H), 7.98-7.94 (m, 1H, aryl H), 7.53-7.46 (m, 2H, aryl H), 7.37-7.27 (m, 1H, aryl H), 7.16-7.14 (m, 2H, aryl H), 6.77-6.72 (m, 2H, aryl H), 6.45 (dd, 1H, J₁=3.24 Hz, J₂=5.64 Hz, aryl H), 6.25 (d, 1H, J=1.16 Hz, aryl H), 5.17 (s, 2H, —CH₂—O—), 4.27 (s, 3H, —NH—CH₂—), 3.90 (s, 2H, —NH₂)

¹³C-NMR (CDCl₃) δ (ppm): 188.46 (C¹¹), 163.49 (C^{4a}), 154.13 (C³), 145.12 (C^{2′}), 140.64 (C^{6a}), 135.46 (C^{10a}), 133.93 (C⁸), 131.94 (C¹), 129.97 (C^{6′}), 129.51 (C¹⁰), 129.25 (C⁹), 128.97 (C^{4′}), 127.44 (C⁷), 121.36 (C^{1′}), 118.64 (C^{5′}), 116.67 (C^{11a}), 116.05 (C^{3′}), 109.24 (C²), 100.49 (C⁴), 73.64 (C⁶), 45.60 (—NH—CH₂—)

IR (ATR) (cm⁻¹): 1626, 1591, 1564, 1495, 1458, 1302, 1262, 1233, 1156, 1121, 750, 703

GC (method 4) 34.0 min

MS m/z (%): 330 (57.6), 225 (40.2), 196 (9.8), 152 (5.4), 106 (100.0), 77 (8.2).

### Example 13

### 3-(2-Aminoethylamino)-6,11-dihydrodibenzo[b,e]oxepin-11-one (10)

In 4.50 g (4.38 mmol) of 5c is added in portions to 4.50 g (74.9 mmol) of ethylenediamine heated to about 120° C. After the addition is complete, the mixture is refluxed for 4 h. Ice-water is added to the mixture, and the resulting precipitate is filtered off and recrystallized from methanol/water. A pale brown powder is obtained.⁵

Yield: 660 mg (56.2%); Melting point: 113-115° C.

¹H-NMR (CDCl₃) δ (ppm): 8.13 (d, 1H, J=4.42 Hz, aryl H), 7.95 (d, 1H, J=3.10 Hz, aryl H), 7.50-7.26 (m, 3H, aryl H), 6.38 (d, 1H, J=4.12 Hz, aryl H), 6.12 (s, 1H, aryl H), 5.14 (s, 2H, —CH₂—O—), 4.81 (s, 1H, >NH), 3.22 (d, 2H, J=2.39 Hz, —(NH)—CH₂—), 2.96 (s, 2H, —CH₂—(NH₂)), 1.64 (s, 2H, —NH₂)

¹³C-NMR (CDCl₃) δ (ppm): 188.18 (C¹¹), 163.57 (C^{4a}), 154.38 (C³), 140.68 (C^{6a}), 135.55 (C^{10a}), 133.91 (C⁸), 131.77 (C¹), 129.51 (C¹⁰), 128.88 (C⁹), 127.35 (C⁷), 116.06 (C^{11a}), 109.02 (C²), 99.73 (C⁴), 73.59 (C⁶), 45.38 (C^{1′}), 29.56 (C^{2′})

IR (ATR) (cm⁻¹): 1591, 1561, 1527, 1391, 1310, 1277, 1233, 1124, 823, 764

GC (method 2) 36.6 min

MS m/z (%): 268 (36.3), 238 (100.0), 225 (13.0), 210 (25.0), 207 (14.9), 196 (8.0), 181 (13.6), 152 (28.7).

### Example 14

### 3-(cis-2-Aminocyclohexylamino)-6,11-dihydrodibenzo[b,e]oxepin-11-one (11a)

By the method described for 10, 1.94 g (17.0 mmol) of cis-1,2-diaminocyclohexane and 0.23 g (1.00 mmol) of 5c are employed, and a dark brown powder is obtained.

Yield: 140 mg (43.4%); Melting point: 116-118° C.

¹H-NMR (CDCl₃) δ (ppm): 8.12 (d, 1H, J=4.46 Hz, aryl H), 7.95 (d, 1H, J=3.42 Hz, aryl H), 7.57-7.44 (m, 2H, aryl H), 7.33-7.27 (m, 1H, aryl H), 6.37 (d, 1H, J=4.35 Hz, aryl H), 6.11 (s, 1H, aryl H), 5.13 (s, 2H, —CH₂—O—), 4.98 (s, 1H, >NH), 3.43 (s, 1H, C1H), 3.15 (s, 1H, C^{2′}H), 1.72 (s, 4H, C^{3′}H₂ und C^{6′}H₂), 1.43 (s, 4H, C⁴H₂ and C⁵H₂) ¹³C-NMR (CDCl₃) δ (ppm): 188.01 (C¹¹), 163.63 (C^{4a}), 153.63 (C³), 140.71 (C^{6a}), 135.55 (C^{10a}), 133.97 (C⁸), 131.71 (C¹), 129.42 (C¹⁰), 128.85 (C⁹), 127.33 (C⁷), 115.60 (C^{11a}), 109.37 (C²), 99.72 (C⁴), 73.56 (C⁶), 53.30 (C^{1′}), 49.11 (C^{2′}), 32.49 (C^{3′}), 29.57 (C^{6′}), 23.21 (C^{5′}), 20.18 (C^{4′})

IR (ATR) (cm⁻¹): 2924, 1628, 1591, 1564, 1520, 1450, 1300, 1277, 1252, 1233, 1156, 1117, 756,702

GC (method 2) 50.5 min

MS m/z (%): 322 (69.7), 304 (21.1), 292 (17.6), 278 (10.5), 264 (26.9), 252 (17.8), 238 (41.4), 226 (100.0), 210 (13.4), 181 (12.9), 152 (22.8), 97 (42.9).

### Example 15

### 3-((1R)-trans-2-Aminocyclohexylamino)-6,11-dihydrodibenzo[b,e]oxepin-11-one (11b)

By the method described for 9, 1.94 g (17.0 mmol) of (1R)-trans-1,2-diaminocyclohexane and 0.23 g (1.00 mmol) of 5c are reacted, and a reddish brown powder is obtained.

Yield: 210 mg (65.1%); Melting point: 114-116° C.

¹H-NMR (CDCl₃) δ (ppm): 8.11 (d, 1H, J=4.42 Hz, aryl H), 7.95-7.88 (m, 1H, aryl H), 7.60-7.25 (m, 3H, aryl H), 6.42 (d, 1H, J=3.93 Hz, aryl H), 6.18 (s, 1H, aryl H), 5.13 (s, 2H, —CH₂—O—), 4.49 (s, 1H, >NH), 2.94 (s, 3H, —NH₂ und >NH), 2.56 (s, 1H, C^{2′}H), 2.05 (t, 2H, J=12.58 Hz, cyclohexyl-CH₂), 1.70 (d, 2H, J=2.91 Hz, cyclohexyl-CH₂), 1.06 (t, 2H, J=11.12 Hz, cyclohexyl-CH₂), 0.87 (d, 2H, J=2.93 Hz, cyclohexyl-CH₂)

¹³C-NMR (CDCl₃) δ (ppm): 188.12 (C¹¹), 163.45 (C^{4a}), 154.14 (C³), 140.63 (C^{6a}), 135.47 (C^{10a}), 133.99 (C⁸), 131.85 (C¹), 129.46 (C¹⁰), 128.92 (C⁹), 127.42 (C⁷), 115.98 (C^{11a}), 109.34 (C²), 100.15 (C⁴), 73.55 (C⁶), 58.45 (C^{1′}), 55.35 (C^{2′}), 34.23 (C^{3′}), 31.97 (C^{6′}), 29.59 (C^{5′}), 24.73 (C^{4′})

IR (ATR) (cm⁻¹): 2924, 1628, 1591, 1564, 1524, 1452, 1299, 1274, 1250, 1157, 1115, 756, 702

GC (method 2) 49.7 min

MS m/z (%): 322 (59.6), 304 (20.6), 292 (16.6), 278 (10.1), 264 (26.9), 262 (11.8), 252 (17.3), 238 (40.7), 226 (100.0), 210 (13.1), 181 (13.4), 152 (24.1), 97 (48.2), 69 (11.0), 56 (10.3).

### Example 16

### 3-((1S)-trans-2-Aminocyclohexylamino)-6,11-dihydrodibenzo[b,e]oxepin-11-one (11c)

By the method described for 9, 1.94 g (17.0 mmol) of (1S)-trans-1,2-diaminocyclohexane and 0.23 g (1.00 mmol) of 5c are reacted, and a reddish brown powder is obtained.

Yield: 150 mg (46.5%); Melting point: 115-117° C.

¹H-NMR (CDCl₃) δ (ppm): 8.12 (d, 1H, J=4.46 Hz, aryl H), 7.96-7.91 (m, 1H, aryl H), 7.55-7.28 (m, 3H, aryl H), 6.41 (dd, 1H, J₁=3.52 Hz, J₂=5.52 Hz, aryl H), 6.18 (d, 1H, J=0.93 Hz, aryl H), 5.13 (s, 2H, —CH₂—O—), 4.41 (d, 1H, J=4.14 Hz, >NH), 3.11 (d, 1H, J=3.66 Hz, C1H), 2.69-2.57 (m, 3H, —NH₂ and C^{2′}H), 2.06 (t, 2H, J=11.44 Hz, cyclohexyl-CH₂), 1.72 (d, 2H, J=2.87 Hz, cyclohexyl-CH₂), 1.06 (t, 2H, J=14.18 Hz, cyclohexyl-CH₂), 0.84 (d, 2H, J=2.44 Hz, cyclohexyl-CH₂)

¹³C-NMR (CDCl₃) δ (ppm): 188.13 (C¹¹), 163.46 (C^{4a}), 154.14 (C³), 140.65 (C^{6a}), 135.48 (C^{10a}), 134.00 (C⁸), 131.84 (C¹), 129.48 (C¹⁰), 128.92 (C⁹), 127.41 (C⁷), 116.02 (C^{11a}), 109.33 (C²), 100.18 (C⁴), 73.57 (C⁶), 58.63 (C^{1′}), 55.44 (C^{2′}), 34.41 (C^{3′}), 32.03 (C^{6′}), 29.60 (C^{5′}), 24.77 (C^{4′})

IR (ATR) (cm⁻¹): 2924, 1628, 1592, 1569, 1525, 1452, 1299, 1275, 1248, 1157, 1137, 1116, 755, 701

GC (method 2) 49.5 min

MS m/z (%): 322 (60.2), 304 (20.1), 292 (16.8), 278 (10.0), 264 (26.7), 262 (12.0), 252 (17.2), 238 (40.7), 226 (100.0), 210 (12.8), 181 (12.3), 152 (22.7), 97 (45.2).

### Example 17

### 3-(2-Acetamidoanilino)-6,11-dihydrodibenzo[b,e]oxepin-11-one (12)

0.50 g (1.58 mmol) of 7b, 1 ml of acetic anhydride and 1 ml of pyridine are refluxed at 100° C. for about 8 h. Ice-water is then added to the mixture, and the resulting precipitate is filtered off. A pale brown powder is obtained by recrystallization from ethanol/water.

Yield: 280 mg (49.4%); Melting point: 168° C.

¹H-NMR (DMSO-d₆) δ (ppm): 9.44 (s, 1H, -Ph-NH—CO—), 8.28 (s, 1H, Ph-NH-Ph), 8.07-7.97 (m, 1H, aryl H), 7.89-7.80 (m, 1H, aryl H), 7.70-7.45 (m, 4H, aryl H), 7.37-7.29 (m, 1H, aryl H), 7.23-7.10 (m, 2H, aryl H), 6.64 (dd, 1H, J₁=3.45 Hz, J₂=5.42 Hz, aryl H), 6.30 (d, 1H, J=0.94 Hz, aryl H), 5.19 (s, 2H, —CH₂—O—), 2.01 (s, 3H, —CO—CH₃)

IR (ATR) (cm⁻¹): 1589, 1573, 1515, 1453, 1296, 1277, 1254, 1228, 1120, 754.

### Example 18

### 1-Ethyl-3-[2-(1-oxo-6,11-dihydrodibenzo[b,e]oxepin-3-ylamino)phenyl]urea (13)

0.50 g (1.58 mmol) of 7b and 0.12 g (1.74 mmol) of ethyl isocyanate are dissolved in 6 ml of dichloromethane and stirred at room temperature for about 5 h. The mixture is evaporated and, after a purification by column chromatography, recrystallized from methanol. The reddish brown product is obtained in this way.⁶

Yield: 100 mg (16.3%); Melting point: 135° C.

¹H-NMR (DMSO-d₆) δ (ppm): 9.26 (s, 1H, -Ph-NH—CO—), 8.41 (s, 1H, CO—NH-Et), 8.07-7.78 (m, 3H, aryl H), 7.65-7.44 (m, 3H, aryl H), 7.21-7.09 (m, 2H, aryl H), 7.01-6.91 (m, 1H, aryl H), 6.54 (dd, 1H, J₁=3.36 Hz, J₂=5.62 Hz, aryl H), 6.12 (d, 1H, J=1.10 Hz, aryl H), 5.17 (s, 2H, —CH₂—O—), 4.04 (s, 1H, Ph-NH-Ph), 3.08 (quint, 2H, J=7.2 Hz, >N—CH₂—), 1.00 (t, 3H, J=7.22 Hz, —CH₃)

IR (ATR) (cm⁻¹): 1590, 1550, 1509, 1447, 1297, 1276, 1250, 1226, 1154, 1119, 752.

### Example 19

### Ethyl [2-(11-oxo-6,11-dihydro-dibenzo[b,e]oxepin-3-ylamino)phenyl]carbamate (14)

0.50 g (1.58 mmol) of 7b and 0.17 g (1.58 mmol) of ethyl chloroformate are stirred with 0.15 g of pyridine at room temperature for about 8 h. Ice-water is added to the mixture, and the resulting precipitate is filtered off. The orange-red product is obtained by purification by column chromatography.

Yield: 150 mg (24.4%); Melting point: 125° C.

¹H-NMR (DMSO-d₆) δ (ppm): 8.74 (s, 1H, -Ph-NH—CO—), 8.29 (s, 1H, Ph-NH-Ph), 8.07-7.96 (m, 1H, aryl H), 7.82-7.78 (m, 1H, aryl H), 7.65-7.49 (m, 4H, aryl H), 7.29-7.26 (m, 1H, aryl H), 7.18-7.13 (m, 2H, aryl H), 6.62 (dd, 1H, J₁=3.35 Hz, 5.57 Hz, aryl H), 6.24 (d, 1H, J=1.10 Hz, aryl H), 5.18 2H, —CH₂—O—), 4.07 (q, 2H, J₁=3.54 Hz, J₂=10.59 Hz, —CO—O—CH₂—), 1.16 (t, 3H, J=7.14 Hz, —CH₃)

IR (ATR) (cm⁻¹): 1589, 1572, 1522, 1454, 1297, 1277, 1221, 1121, 61, 757.

### Example 20

### 3-(2-Methylaminophenylamino)-6,11-dihydrodibenzo[b,e]oxepin-11-one (15)

0.15 g (0.47 mmol) of 7b is dissolved in 20 ml of DMSO. 0.14 g (0.98 mmol) of methyl iodide and 3.78 g (27.4 mmol) of potassium carbonate are added to the solution. The mixture is stirred at 10° C. for 3 h. After cooling to room temperature, ice-water is added thereto, and the resulting precipitate is filtered off. The crude product obtained in this way is purified by HPLC on RP18 silica gel with acetonitrile/water 6+4, and the yellowish product is obtained.

¹H-NMR (CDCl₃) δ (ppm): 8.23-8.12 (m, 1H, aryl H), 7.99-7.92 (m, 1H, aryl H), 7.54-7.41 (m, 3H, aryl H), 7.36-7.26 (m, 2H, aryl H), 7.17-6.98 (m, 1H, aryl H), 6.85-6.79 (m, 1H, aryl H), 6.53-6.47 (m, 1H, aryl H), 6.18-6.16 (m, 1H, aryl H), 5.15 (s, 2H, —CH₂—O—), 3.28 (s, 1H, >NH), 3.05 (s, 4H, —NH—CH₃)

¹³C-NMR (CDCl₃) δ (ppm): 188.42 (C¹¹), 163.04 (C^{4a}), 155.40 (C³), 143.22 (C^{2′}), 140.71 (C^{6a}), 135.66 (C^{10a}), 133.81 (C⁸), 131.74 (C¹), 129.54 (C¹⁰), 128.88 (C⁹), 128.39 (C⁷), 128.25 (C^{5′}), 127.42 (C^{4′}), 127.38 (C^{1′}), 119.38 (C^{6′}), 116.36 (C^{3′}), 115.03 (C^{11a}), 107.30 (C²), 99.80 (C⁴), 73.63 (C⁶), 39.86 (-Me)

IR (ATR) (cm⁻¹): 1627, 1591, 1564, 1537, 1384, 1300, 1283, 1255, 1235, 1203, 1159, 1116, 1102, 755, 701

GC (method 4) 37.7 min

MS m/z (%): 330 (100.0), 315 (15.7), 301 (10.5), 287 (8.0), 285 (7.1), 197 (6.0), 181 (6.1), 159 (13.4), 152 (6.2).

### Example 21

### 3-(2,4-Difluorophenylamino)-6,11-dihydrodibenzo[b,e]oxepin-11-one (16a)

By general method A, 0.10 g (2.30 mmol) of sodium hydride (55%), 0.29 g (2.21 mmol) of 2,4-difluoroaniline and 0.50 g (2.19 mmol) of 5c are employed with use of 5 ml of dimethylformamide. The mixture is refluxed for about 8 h. Working up takes place with about 50 ml of ice-water without acidification. The precipitate is filtered off and purified by MPLC on an RP18 silica gel column with acetonitrile/water 6+4 as mobile phase.

Yield: 50 mg (6.8%)

¹H-NMR (CDCl₃) δ (ppm): 8.30-8.17 (m, 2H, aryl H), 7.95 (d, 1H, J=3.72 Hz, aryl H), 7.78-7.70 (m, 1H, aryl H), 7.59-7.29 (m, 2H, aryl H), 6.98-6.81 (m, 2H, aryl H), 6.67-6.62 (m, 1H, aryl H), 6.48 (s, 1H, aryl H), 5.93 (s, 1H, >NH), 5.16 (s, 2H, —CH₂—O—)

¹³C-NMR (CDCl₃) δ (ppm): 188.65 (C¹¹), 184.28, 181.52, 163.12 (C^{4a}), 153.70, 150.22 (C³), 140.50 (C^{6a}), 135.35 (C^{10a}), 134.82, 134.29, 134.12 (C⁸), 133.83, 133.64, 132.86, 132.16 (C¹), 129.65, 129.49 (C¹⁰), 129.06 (C⁹), 127.52 (C⁷), 126.79, 124.20, 124.06, 118.38 (C^{11a}), 115.68, 111.57, 111.50, 111.13, 111.06, 110.36 (C²), 108.32, 105.19, 104.66, 104.19, 103.17 (C⁴), 73.59 (C⁶)

IR (ATR) (cm⁻¹): 1667, 1629, 1588, 1575, 1552, 1523, 1500, 1479, 1457, 1436, 1376, 1360, 1348, 1329, 1307, 1288, 1261, 1231, 1219, 1181, 1157, 1142, 1121, 1097, 1062, 1028, 965, 926, 849, 827, 761, 720, 711, 704

GC (method 3) 24.5 min

MS m/z (%): 337 (100), 308 (22.8), 288 (3.8), 280 (2.7), 209 (4.3), 181 (12.4), 152 (11.6), 139 (2.9), 128 (3.1), 115 (3.2), 89 (5.5), 63 (4.3).

### Example 22

### 3-(2,4-Dichlorophenylamino)-6,11-dihydrodibenzo[b,e]oxepin-11-one (16b)

By general method A, 0.05 g (1.15 mmol) of sodium hydride (55%), 0.18 g (1.11 mmol) of 2,4-dichloroaniline and 0.25 g (1.10 mmol) of 5c are employed with use of 2.5 ml of dimethylformamide. The mixture is refluxed overnight. Working up takes place with about 30 ml of ice-water without acidification. The precipitate is filtered off and purified by MPLC on an RP18 silica gel column with acetonitrile as mobile phase.

¹H-NMR (CDCl₃) δ (ppm): 8.37-8.19 (m, 1H, aryl H), 8.00-7.89 (m, 1H, aryl H), 7.61-7.32 (m, 5H, aryl H), 7.29-7.17 (m, 1H, aryl H), 6.81-6.74 (m, 1H, aryl H), 6.70-6.64 (m, 1H, aryl H), 6.27 (s, 1H, >NH), 5.19 (s, 2H, —CH₂—O—)

¹³C-NMR (CDCl₃) δ (ppm): 188.73 (C¹¹), 162.95 (C^{4a}), 148.45 (C³), 140.45 (C^{6a}), 136.06 (C^{1′}), 135.31 (C^{10a}), 134.12 (C⁸), 132.29 (C¹), 129.65 (C^{3′}), 129.51 (C¹⁰), 129.14 (C⁹), 127.59 (C⁷ and C^{4′}), 127.16 (C^{2′}), 125.01 (C^{5′}), 120.54 (C^{6′}), 119.24 (C^{11a}), 111.68 (C²), 105.20 (C⁴), 73.60 (C⁶)

IR (ATR) (cm⁻¹): 1589, 1573, 1514, 1468, 1326, 1298, 1278, 1253, 1121, 1100, 758, 703

GC (method 3) 41.9 min

MS m/z (%): 373 (12.0), 371 (66.1), 369 (100.0), 344 (1.9), 342 (9.4), 340 (13.8), 299 (14.3), 270 (7.9), 243 (7.1), 241 (7.6), 209 (5.7), 181 (17.6), 152 (18.2), 135 (9.7), 120 (10.6), 89 (11.4), 63 (6.5).

### Example 23

### 3-(2,4-Dibromophenylamino)-6,11-dihydrodibenzo[b,e]oxepin-11-one (16c)

By general method A, 0.10 g (2.29 mmol) of sodium hydride (55%), 0.55 g (2.21 mmol) of 2,4-dibromoaniline and 0.50 g (2.19 mmol) of 5c are employed with use of 5 ml of dimethylformamide. The mixture is refluxed for about 8 h. Working up takes place with about 30 ml of ice-water without acidification. The precipitate is filtered off and purified by MPLC on an RP18 silica gel column with acetonitrile as mobile phase to result in the yellowish product.

Yield: 80 mg (8.0%); Melting point: 157.2-159.2° C.

¹H-NMR (CDCl₃) δ (ppm): 8.31-8.20 (m, 1H, aryl H), 7.98-7.89 (m, 1H, aryl H), 7.86-7.69 (m, 1H, aryl H), 7.60-7.29 (m, 5H, aryl H), 6.88-6.75 (m, 1H, aryl H), 6.70-6.62 (m, 1H, aryl H), 6.26 (s, 1H, >NH), 5.19 (s, 2H, —CH₂—O—)

¹³C-NMR (CDCl₃) δ (ppm): 188.74 (C¹¹), 162.95 (C^{4a}), 148.36 (C³), 140.44 (C^{6a}), 137.74 (C^{1′}), 135.31 (C^{10a} and C^{3′}), 134.13 (C⁸), 132.30 (C¹), 131.15 (C^{5′}), 129.50 (C¹⁰), 129.14 (C⁹), 127.59 (C⁷), 120.89 (C^{6′}), 119.32 (C^{11a}), 115.47 (C^{4′}), 114.86 (C^{2′}), 111.75 (C²), 105.34 (C⁴), 73.60 (C⁶)

IR (ATR) (cm⁻¹): 1634, 1602, 1586, 1561, 1463, 1329, 1301, 1276, 1252, 1121, 1049, 818, 759, 702, 686

GC (method 3) 62.0 min

MS m/z (%): 461 (51.3), 459 (100.0), 457 (51.1), 432 (4.6), 430 (8.6), 428 (4.3), 299 (24.8), 270 (21.9), 254 (7.0), 241 (15.0), 181 (16.0), 152 (16.3), 150 (17.5), 135 (11.8), 121 (16.1), 90 (14.5), 89 (14.3), 63 (8.4).

### Example 24

### 3-(4-fluoro-2-nitrophenylamino)-6,11-dihydrodibenzo[b,e]oxepin-11-one

### a) 2-(3-Fluorophenoxy)methylbenzoic Acid

2.30 g (52.7 mmol) of sodium hydride (55%) are suspended in 50 ml of dimethylformamide. Then 5.61 g (50.0 mmol) of 3-fluorophenol are added. After gas evolution ceases, 6.80 g (50.7 mmol) of phthalide are added, and the mixture is refluxed at about 160° C. for about 5 h. After the reaction mixture has cooled, 90 ml of ice-water are added, and 20% strength hydrochloric acid is used to acidify. The resulting precipitate is filtered off and washed with 10% strength hydrochloric acid and dried over calcium chloride.

Yield: 4.37 g (48.5%); melting point: 90-92° C.

¹H NMR (CDCl₃) δ(ppm): 8.19-7.25 (m, 7H, aryl H), 6.80-6.67 (m, 1H, aryl H), 5.34 (s, 2H, —CH₂—O—)

¹³C-NMR (CDCl₃) δ(ppm): 171.16 (d, J=10.40 Hz, —COOH), 163.54 (d, J=243.60 Hz, C^{1′}), 159.86 (d, J=10.86 Hz, C^{3′}), 143.19 (d, J=323.10 Hz, C²), 132.61 (d, J=92.76 Hz, C⁴), 131.43 (d, J=248.16 Hz, C^{5′}), 130.16 (d, J=9.96 Hz, C⁶), 127.31 (d, J=9.46 Hz, C⁵), 125.95 (d, J=29.06 Hz, C¹), 123.84 (d, J=187.00 Hz, C³), 110.56 (d, J=2.96 Hz, C^{6′}), 107.76 (d, J=21.16 Hz, C^{4′}), 102.65 (d, J=24.70 Hz, C^{2′}), 68.92 (d, J=64.56 Hz, —CH₂—O—)

IR (ATR (cm⁻¹): 1690, 1613, 1595, 1581, 1490, 1311, 1287, 1273, 1165, 1139, 1042, 999, 957, 755, 735, 679, 671.

### b) 3-Fluoro-6,11-dihydrodibenzo[b,e]oxepin-11-one

5.00 g (20.3 mmol) of 2-(3-fluorophenoxy)methylbenzoic acid are dissolved in 25 ml of sulfolane under argon by heating to about 100° C. Then 48.5 ml (100 g) of polyphosphoric acid are added, and the mixture is stirred at 100° C. for 2 h. After the reaction is complete, about 150 ml of ice-water are added, and the mixture is stirred at room temperature overnight. The precipitated product is filtered off and dried over calcium chloride.

Yield: 2.30 g (49.7%); melting point: 79-81° C.

¹H NMR (CDCl₃) δ(ppm): 8.28 (dd, 1H, J₁=9.00 Hz, J₂=6.80 Hz, aryl H), 7.90 (dd, 1H, J₁=7.60 Hz, J₂=1.40 Hz, aryl H), 7.62-7.44 (m, 2H, aryl H), 7.37 (dd, 1H, J₁=7.10 Hz, J₂=1.30 Hz, aryl H), 6.90-6.71 (m, 2H, aryl H), 5.21 (s, 2H, —CH₂—O—)

¹³C-NMR (CDCl₃) δ(ppm): 189.52 (C¹¹), 165.96 (d, J=332.90 Hz, C³), 163.54 (d, J=65.36 Hz, C^{4a}), 140.24 (C^{6a}), 135.00 (C^{10a}), 134.38 (C⁸), 133.66 (d, J=94.00 Hz, C¹), 129.44 (C¹⁰), 129.31 (C⁹), 127.74 (C⁷), 122.17 (d, J=2.64 Hz, C^{11a}), 110.33 (d, J=21.80 Hz, C²), 106.86 (d, J=23.70 Hz, C⁴), 73.76 (C⁶)

IR (ATR) (cm⁻¹): 1643, 1611, 1596, 1576, 1296, 1242, 1208, 1138, 1115, 1104, 1023, 851, 753, 695

GC (method 1) 18.8 min

MS m/z (%): 228 (100.0), 199 (73.6), 170 (24.9), 89 (13.6).

### c) 3-(4-Fluoro-2-nitrophenylamino)-6,11-dihydrodibenzo[b,e]oxepin-11-one

By the method described under a), 0.10 g (2.30 mmol) of sodium hydride (55%), 0.34 g (2.21 mmol) of 2-fluoro-4-nitroaniline and 0.50 g (2.19 mmol) of 3-fluoro-6,11-dihydrodibenzo[b,e]oxepin-11-one are employed using 4 ml of dimethylformamide. The mixture is refluxed at about 80° C. for about 8 h. Working up takes place with about 50 ml of ice-water without acidification. The crude product is extracted with ethyl acetate, the solvent is removed in a rotary evaporator, and the residue is recrystallized from ethanol.

Yield: 320 mg (40.1%)

¹H NMR (CDCl₃) δ(ppm): 8.37-8.24 (m, 2H, aryl H), 7.97-7.90 (m, 2H, aryl H), 7.61-7.49 (m, 3H, aryl H), 7.40-7.36 (m, 1H, aryl H), 6.97-6.87 (m, 2H, aryl H), 5.22 (s, 2H, —CH2-O—), >NH not detected

¹³C-NMR (CDCl₃) δ(ppm): 189.02 (C¹¹), 162.58 (C^{4a}), 152.56 (C^{4′}), 145.96 (C^{2′}), 152.64 (C³), 140.31 (C^{6a}), 135.19 (C^{10a}), 134.09 (C⁸), 132.57 (C¹), 129.51 (C¹⁰), 129.27 (C⁹), 127.72 (C⁷), 127.16 (C^{1′}), 123.46 (C^{5′}), 121.25 (C^{11a}), 119.82 (C^{6′}), 114.61 (C²), 112.23 (C^{3′}), 109.92 (C⁴), 73.65 (C⁶)

IR (ATR) (cm⁻¹): 1607, 1588, 1509, 1461, 1421, 1310, 1298, 1261, 1244, 1229, 1212, 1187, 1154, 1122, 1060, 1025, 933, 924, 821, 758, 697.

### d) 3-(2-Amino-4-fluorophenylamino)-6,11-dihydrodibenzo[b,e]oxepin-11-one

0.25 g (0.69 mmol) of 3-(4-fluoro-2-nitrophenylamino)-6,11-dihydrodibenzo-[b,e]oxepin-11-one is dissolved in 5 ml of ethanol, and 0.78 g (3.44 mmol) of tin(II) chloride dihydrate is added, and the mixture is stirred at 70° C. for about 2 h. After the mixture has cooled to room temperature, ice-water is added and sodium hydroxide solution is used to make alkaline. The aqueous phase is extracted with ethyl acetate, the ethyl acetate phase is washed with saturated sodium chloride solution, and the solvent is removed. The resulting crude product is purified by MPLC on an RP18 silica gel column initially with acetonitrile/water 6+4 and then with pure acetonitrile as mobile phase.

Yield: 60 mg (26.0%); melting point: 182.6° C.

¹H NMR (CDCl₃) δ(ppm): 8.16 (d, 1H, J=8.96 Hz, aryl H), 7.93 (d, 1H, J=7.04 Hz, aryl H), 7.52-7.39 (m, 2H, aryl H), 7.31 (d, 1H, J=7.00 Hz, aryl H), 7.09-7.01 (m, 1H, aryl H), 6.54-6.39 (m, 3H, aryl H), 6.11 (s, 1H, aryl H), 5.59 (s, 1H, >NH), 5.12 (s, 2H, —CH₂—O—), 3.20 (ws, 2H, —NH₂)

¹³C-NMR (CDCl₃) δ(ppm): 188.63 (C¹¹), 163.36 (C^{4a}), 162.16 (d, J=2.42.50 Hz, C^{4′}), 152.64 (C³), 144.90 (d, J=11.30 Hz, C^{2′}), 140.57 (C^{6a}), 135.37 (C^{10a}), 134.16 (C⁸), 132.02 (C¹), 129.44 (C¹⁰), 129.32 (d, J=12.10 Hz, C^{6′}), 128.99 (C⁹), 127.47 (C⁷), 120.72 (C^{1′}), 117.43 (C^{11a}), 109.43 (C²), 105.35 (d, J=22.60 Hz, C^{5′}), 102.55 (d, J=25.54 Hz, C^{3′}), 101.83 (C⁴), 73.54 (C⁶)

IR (ATR) (cm⁻¹): 1625, 1591, 1565, 1508, 1470, 1303, 1275, 1255, 1232, 1157, 1120, 975, 923, 842, 758, 701

GC (method 4) 35.21 min

MS m/z (%): 334 (100.0), 319 (12.3), 317 (8.1), 315 (5.3), 305 (10.6), 303 (2.5), 291 (6.4), 287 (6.0), 277 (2.4), 275 (3.1), 263 (2.3), 199 (3.8), 187 (4.3), 181 (5.3), 163 (3.9), 158 (4.7), 152 (6.3), 150 (3.3), 144 (3.1), 137 (3.3), 128 (2.2), 125 (3.5), 115 (2.7), 98 (3.7), 89 (4.5), 83 (2.7), 63 (2.4).

B. Preparation of Compounds of the Formula I in which X—Y is CH₂—CH₂ or CH═CH

### Example 25

### 2-(2-Aminophenylamino)-10,11-dihydrodibenzo[a,d]cycloheptan-5-one (30)

### a) Methyl 2-bromomethylbenzoate (21)

60.0 g (0.4 mol) of methyl o-toluate 20 are dissolved by stirring in 375 ml of CHCl₃ in a dry 500 ml three-necked flask, and 75.0 g (0.42 mol) of N-bromosuccinimide and 0.75 g of azaisobutyronitrile are added. The mixture is cautiously heated to about 65° C. with a reflux condenser until the reaction starts, and then the heating source is removed and, after the reaction has subsided, the mixture is refluxed at 70° C. for 5 h. On cooling to RT (room temperature), succinimide precipitates and is filtered off. Concentration of the filtrate results in the crude product which is used further without workup.

GC (method 5) 7.3 min

MS m/z (%): 230/228 (21, M⁺), 199/197 (22), 149 (100, M⁺-Br), 118 (41), 91 (54, tropylium⁺).

### b) (2-Methoxycarbonylbenzyl)triphenylphosphonium Bromide (22)

40.0 g (0.175 mol) of methyl 2-bromomethylbenzoate 21 are dissolved in 300 ml of acetone in a dry 500 ml round-bottomed flask and stirred at RT for 15 min. Addition of 52.0 g (0.2 mol) of triphenylphosphine is followed by refluxing at about 60° C. for 5 h. The title compound precipitates on cooling and is filtered off and, after washing with acetone, dried in vacuo.⁷

Yield: 51.9 g (60.5%); Melting point: decomposition above 234° C.

IR (ATR): 1711 (COOR), 1696, 1585, 1438, 1273, 1109, 753, 716, 706, 689 cm⁻¹.

### c) Methyl (E/Z)-3′-nitrostilbene-2-carboxylate (24)

The stilbene 24 is synthesized by general method D by using 16.8 g of sodium methanolate solution (30% in MeOH) in 280 ml of methanol, 40.0 g (81.4 mmol) of phosphonium salt 22 and 12.8 g (84.7 mmol) of 3-nitrobenzaldehyde 23. Refluxing time 6 h, extraction of the blackish red oily residue after taking up in water with 3×100 ml of Et₂O.

Yield: 18.0 g (78.1%); Melting point: 66.5° C.

GC (method B) (E-) 9.7 min, (Z-) 12.8 min

MS (E-) m/z (%): 283 (100, M⁺), 266 (22), 251 (31), 236 (24), 205 (21), 194 (14), 176 (43), 165 (26), 151 (15), 76 (13, C₆H₄).

(Z-) m/z (%): 283 (100, M⁺), 266 (20), 251 (28), 236 (22), 205 (18), 194 (13), 176 (36), 165 (21), 151 (12), 76 (11, C₆H₄).

IR (ATR); 1714 (COOR), 1525, 1435, 1348, 1295, 1267, 1252, 1188, 1130, 1079, 956, 747, 719, 704, 672 cm⁻¹.

### d) (E/Z)-3′-Nitrostilbene-2-carboxylic Acid (25)

The ester 24 is cleaved by general method E by using 16.0 g (56.5 mmol) of the ester, 150 ml of MeOH and 75 ml of 20% strength sodium hydroxide solution. Refluxing time 5.5 h, extraction with 3×100 ml of CH₂Cl₂. The crude product is precipitated with concentrated HCl in the form of a yellowish precipitate, which is filtered off. Purification takes place by digestion with diethyl ether and concentration of the filtrate to dryness.

Yield: 12.2 g (80.0%); Melting point: 164-166° C.

IR (ATR): 1682 (COOH), 1519, 1349, 1304, 1268, 1249, 1077, 907, 754, 733, 716 cm⁻¹.

### e) 2-[2-(3-Aminophenyl)ethyl]benzoic Acid⁸ (26)

10.0 g (37.1 mmol) of the nitro compound 25 are slowly dissolved in 150 ml of EtOAc (ethyl acetate) in a hydrogenation vessel and then 1.0 g of Pd/C (10%) is stirred in, the apparatus is closed and, after flushing with H₂ several times, a constant pressure of 4 bar is applied. After 10 h, the title compound 26 is isolated by filtering off the Pd-carbon and distilling off the solvent.

Melting point: 114° C.

GC 10.5 min (method 6)

MS m/z (%): 241 (77, M⁺), 223 (14, M⁺-H₂O), 208 (8), 194 (7), 106 (100, NH₂-Ph-CH₂⁺), 77 (17, C₆H₄).

### f) 2-[2-(3-Acetamidophenyl)ethyl]benzoic Acid⁸ (27)

The residue obtained in stage (e) is mixed with 25 ml of acetic anhydride and stirred at RT for 15 h. 200 ml of ice-water are added to the mixture, and it is extracted repeatedly with 200 ml of EtOAc. The combined organic phases are concentrated to about 50 ml and reextracted with 2×50 ml of water and then freed of solvent in vacuo. A yellowish oily residue remains, and the product is isolated therefrom by taking up in a little MeOH, precipitating with ice-water and filtering.

Yield: 8.3 g (78.9%) over 2 stages; Melting point: 145-148° C.

¹H-NMR (DMSO-d6) δ in ppm: 2.02 (s, 3H, CH₃), 2.73-3.20 (m, 4H, CH₂—CH₂), 6.9 (d, 1H, 7.6 Hz), 7.18 (t, 1H, 8.2 Hz), 7.29-7.33 (m, 2H), 7.40-7.48 (m, 3H), 7.81 (dd, 1H, 3.5 Hz), 9.86 (s, 1H, NH), 12.81 (s, 1H, COOH).

IR (ATR): 1690 (C═O), 1664 (amide), 1593 (amide II), 1558, 1489, 1305, 1278, 782, 747, 699 cm⁻¹.

### g) 2-(Acetamido)-10,11-dihydrodibenzo[a,d]cycloheptan-5-one⁸ (28)

By general method F, 3.6 g (12.7 mmol) of the carboxylic acid 27 are dissolved in 40 ml of sulfolane, 100 g of PPA are added, and the reaction mixture is refluxed at 110° C. for 5 h. Hydrolysis with 250 ml of ice-water leads to precipitation of the crude product during stirring at RT for 17 h, and the product is filtered off and purified by washing with H₂O.

Yield: 2.35 g (69.7%); Melting point: 152° C.

¹H-NMR (DMSO-d6) δ (ppm): 2.01 (s, 1H, CH₃), 3.11 (s, 4H, CH₂—CH₂), 7.34 (t, 2H, 7.4 Hz), 7.47 (d, 1H), 7.55 (d, 2H, 8.5 Hz), 7.84 (d, 1H, 7.7 Hz), 7.94 (d, 1H, 8.3 Hz), 10.23 (s, 1H, NH).

GC (method 6) 18.7 min

MS m/z (%): 265 (99, M⁺), 223 (100, aminodibenzosuberone⁺), 208 (9, dibenzosuberone⁺), 194 (70), 178 (13), 165 (22), 152 (12).

IR (ATR): 3351 (C—N), 1689 (C═O), 1627 (amide), 1587 (amide II), 1524, 1290, 1271, 1240, 938, 764, 698 cm⁻¹.

### h) 2-Amino-10,11-dihydrodibenzo[a,d]cycloheptan-5-one*HCl^{8,9} (29)

2.0 g (7.54 mmol) of the acetamide 28 are suspended in 60 ml of 20% strength HCl and refluxed at about 100° C. for 4 h. The resulting precipitate is filtered off after cooling, and the product remains as a sandy-colored powder.

Yield: 1.8 g (91.9%); Melting point: 219° C.

¹H-NMR (DMSO-d6) δ in ppm: 2.96-3.10 (m, 4H, CH₂—CH₂), 5.37 (s, 2H, NH₂), 6.86 (d, 1H, 2.0 Hz), 6.95 (d, 1H, 3.12 Hz), 7.35 (t, 2H, 7.8 Hz), 7.49 (t, 1H, 8.2 Hz), 7.86 (d, 1H, 6.5 Hz), 7.95 (d, 1H, 8.5 Hz).

¹³C-NMR (DMSO-d6) δ in ppm: 34.3 (CH₂), 35.3 (CH₂), 117.0 (C³), 118.7 (C¹), 126.9 (C⁷), 129.4 (C⁹), 130.5 (C⁶), 131.2 (C^{4a}), 132.5 (C⁴), 133.2 (C⁸), 138.8 (C^{5a}), 142.1 (C^{9a}), 145.0 (C^{11a}), 145.1, (C²), 192.2 (C⁵).

GC (method 6) 12.1 min

MS m/z (%): 223 (100, M⁺), 208 (8, dibenzosuberone⁺), 194 (74), 180 (13), 165 (15), 152 (9), 97 (13).

IR (ATR): 2563 (NH₃⁺), 1652 (C═O), 1595, 1510, 1290, 910, 846, 763, 693 cm⁻¹.

### i) 2-(2-Aminophenylamino)-10,11-dihydrodibenzo[a,d]cycloheptan-5-one (30)

0.52 g of NaH (55-60%) are suspended in 12 ml of THF (tetrahydrofuran) in a dry 100 ml three-necked flask with reflux condenser, drying tube and septum, and 1.0 g (3.85 mmol) of the suberone 29 is added. After gas evolution ceases, 0.54 g (3.85 mmol) of 2-fluoronitrobenzene is added dropwise, and the mixture is refluxed at about 150° C. for 17.5 h. This is followed by hydrolysis with 75 ml of ice-water and filtration of the resulting precipitate, which comprises the intermediate 2-(2-nitrophenylamino)-10,11-dihydrodibenzo[a,d]cycloheptan-5-one.

Reduction of this nitro compound (filtration residue about 0.5 g) takes place by general method G using 30 ml of i-PrOH, 15 ml of conc. HCl and 500 mg of tin powder. Basification is followed by extraction with 2×75 ml EtOAc. Purification takes place by column chromatography on SiO₂ with CH₂Cl₂/EtOH (95+5) and subsequent recrystallization from MeOH/H₂O.

Yield: 0.13 g (10.7%); Melting point: 153° C.

¹H-NMR (DMSO-d6) δ in ppm: 2.96-3.07 (m, 4H, CH₂—CH₂), 4.86 (s, 2H, NH₂), 6.46 (d, 1H, 2.1 Hz), 6.58-6.64 (m, 2H), 6.77 (d, 1H, 8.3 Hz), 6.90-7.04 (m, 2H), 7.32 (t, 2H, 8.3 Hz), 7.41-7.46 (m, 1H), 7.84 (d, 1H, 6.4 Hz), 7.94-7.98 (2H, NH+ar).

¹³C-NMR (DMSO-d6) δ in ppm: 34.4 (CH₂), 36.3 (CH₂), 112.2 (C³), 113.2 (C¹), 116.2 (C^{3′}), 117.2 (C^{6′}), 125.5 (C^{1′}), 126.1 (C^{4′}), 126.4 (2 C, C^{4a}+C^{5′}), 126.7 (C⁷), 128.9 (C⁹), 130.6 (C⁶), 132.1 (C⁴), 133.8 (C⁸), 139.5 (C^{5a}), 142.0 (C^{9a}), 143.4 (C^{2′}), 145.7 (C^{11a}), 151.0 (C²), 190.7 (C⁵).

GC (method 6) 29.6 min

MS m/z (%): 314 (100, M⁺), 299 (10, M⁺-NH), 285 (10), 271 (7), 178 (7), 165 (7), 143 (5), 107 (6).

IR (ATR): 1599 (C═O), 1581, 1566, 1499, 1290, 1279, 1258, 1111, 750, 694 cm⁻¹.

### Example 26

### 2-(4-Aminophenylamino)-10,11-dihydrodibenzo[a,d]cycloheptan-5-one (32)

0.39 g of NaH (55-60%) is suspended in 18 ml of THF in a dry 100 ml three-necked flask with reflux condenser, drying tube and septum, and 0.75 g (2.85 mmol) of the suberone 29 are added. After gas evolution ceases, 0.40 g (2.85 mmol) of 4-fluoronitrobenzene is added dropwise, and the mixture is refluxed at about 150° C. for 17 h. This is followed by hydrolysis with 75 ml of ice-water and filtration of the resulting precipitate, which comprises the intermediate 2-(4-nitrophenylamino)-10,11-dihydrodibenzo[a,d]cycloheptan-5-one.

Reduction of this nitro compound (filtration residue about 0.2 g) takes place by general method G using 10 ml of i-PrOH, 5 ml of conc. HCl and 200 mg of tin powder. Basification is followed by extraction with 2×50 ml of EtOAc. Purification takes place by column chromatography on SiO₂ with CH₂Cl₂/EtOH (95+5) and then on SiO₂ with Et₂O.

Yield: 0.05 g (5.5%); Melting point: 217° C.

¹H-NMR (CDCl₃) δ in ppm: 3.06-3.16 (m, 4H, CH₂—CH₂), 3.66 (d, 2H, NH₂), 5.81 (s, 1H, NH), 6.55 (d, 1H, 2.3 Hz), 6.69-6.75 (m, 3H), 7.04 (d, 2H, 6.5 Hz), 7.19 (d, 1H), 7.31-7.41 (m, 2H), 8.02 (d, 1H, 9.3 Hz), 8.15 (d, 1H, 8.7 Hz).

¹³C-NMR (CDCl₃) δ in ppm: 34.7 (CH₂), 36.3 (CH₂), 112.1 (C³), 113.0 (C¹), 115.9 (2 C, C^{3′}+C^{5′}), 125.2 (2 C, C^{2′}+C^{6′}) 126.4 (C⁷), 127.8 (C^{4a}), 128.3 (C⁹), 130.7 (C⁶), 131.1 (C^{1′}), 131.6 (C⁴), 134.1 (C⁸), 139.5 (C^{5a}), 141.6 (C^{9a}), 143.6 (C^{4′}), 145.6 (C^{11a}), 150.0 (C²), 192.9 (C⁵).

GC (method 6) 36.3 min

MS m/z (%): 314 (100, M⁺), 286 (5, M⁺-CO), 178 (5), 165 (5), 143 (5), 107 (10).

IR (ATR): 1582 (C═O), 1562, 1506, 1295, 1281, 1211, 1109, 825, 758 cm⁻¹.

### Example 27

### 2-(2-Aminophenylamino)dibenzo[a,d]cyclohepten-5-one (38)

### a) Methyl (E/Z)-3′-fluorostilbene-2-carboxylate (34)

Compound 34 is synthesized by general method D by using 13.0 g of sodium methanolate solution (30% in MeOH) in 225 ml of methanol, 40.0 g (81.4 mmol) of phosphonium salt 22 and 11.2 g (90.2 mmol) of 3-fluorobenzaldehyde. Refluxing time 7 h, extraction of the yellowish white oily residue after taking up in water with 3×150 ml of Et₂O.

Yield: 16.5 g (79.1%)

GC (method 6) (E-) 5.9 min, (Z-) 7.7 min

MS (E-) m/z (%): 256 (100, M⁺), 241 (5, M⁺-CH₃), 225 (41, M⁺-OCH₃), 197 (83, M⁺-COOCH₃), 177 (29), 170 (16), 161 (11), 98 (15).

(Z-) m/z (%): 256 (100, M⁺), 241 (4, M⁺-CH₃), 225 (40, M⁺-OCH₃), 197 (77, M⁺-COOCH₃), 177 (23), 170 (13), 161 (10), 98 (18).

### b) 3′-Fluorostilbene-2-carboxylic Acid¹¹ (35)

The ester 34 is cleaved by general method E by using 13.0 g (50.7 mmol) of the ester, 70 ml of MeOH and 60 ml of 20% strength sodium hydroxide solution. Refluxing time 6 h, extraction with 2×75 ml of CH₂Cl₂. The crude product is precipitated with concentrated HCl in the form of a yellowish oil which gradually solidifies with stirring to a precipitate which is filtered off. Purification takes place by digestion with Et₂O and concentration of the filtrate to dryness.

Yield: 6.5 g (52.9%); Melting point: 113° C. GC (method 6) (E-) 7.0 min, (Z-), 9.0 min

MS (E-) m/z (%): 242 (100, M⁺), 224 (15, M⁺-H₂O), 196 (68, 224-CO), 177 (22), 133 (42), 98 (11).

(Z-) m/z (%): 242 (100, M⁺), 224 (16, M⁺-H₂O), 196 (68, 224-CO), 177 (21), 133 (48), 106 (53).

IR (ATR): 1675 (C═O), 1582, 1303, 1266, 1219, 929, 890, 777, 768, 758, 738, 706 cm⁻¹.

### c) 2-Fluorodibenzo[a,d]cyclohepten-5-one¹⁰ (36)

By general method F, 6.5 g (26.8 mmol) of the carboxylic acid 35 are dissolved in 65 ml of sulfolane, 130 g of PPA are added, and the mixture is refluxed at 110° C. for 6 h. Hydrolysis with 250 ml of ice-water leads to precipitation of the crude product while stirring at RT for 16 h, and the product is filtered off and washed with H₂O. Purification takes place by column chromatography on SiO₂ with CH₂Cl₂.

Yield: 2.45 g (40.7%); Melting point: 120° C.

¹H-NMR (DMSO-d6) δ in ppm: 7.17-7.34 (m, 2H, CH═CH), 7.48 (t, 1H, 8.8 Hz), 7.60-7.71 (m, 2H), 7.75-7.80 (m, 2H), 8.10-8.21 (m, 2H). GC (method 6) 7.7 min

MS m/z (%): 224 (55 μm⁺), 196 (100, M⁺-Co), 170 (10,196-CH═CH).

IR (ATR): 1644 (C═O), 1606, 1570, 1300, 1251, 955, 797, 730, 686 cm⁻¹.

### d) 2-(2-Aminophenylamino)dibenzo[a,d]cyclohepten-5-one (38)

150 mg of NaH (55-60%) are suspended in 6 ml of dimethylformamide in a dry 100 ml three-necked flask with reflux condenser, drying tube and septum, and 450 mg (3.25 mmol) of 2-nitroaniline 37. After gas evolution ceases, 700 mg (3.12 mmol) of the suberenone 36 are added dropwise, and the mixture is refluxed at about 150° C. for 24 h. This is followed by hydrolysis with 75 ml of ice-water and filtration of the resulting precipitate, which comprises the intermediate 2-(2-nitrophenylamino)dibenzo[a,d]cycloheptan-5-one.

This nitro compound (filtration residue about 0.4 g) is reduced by general method G using 20 ml of i-PrOH, 10 ml of conc. HCl and 400 mg of tin powder. Basification is followed by extraction with 2×75 ml of EtOAc. Purification takes place by column chromatography on SiO₂ with CH₂Cl₂/EtOH (95+5) and subsequent recrystallization from MeOH/H₂O.

Yield: 0.07 g (7.2%); Melting point: 232° C.

¹H-NMR (DMSO-d6) δ in ppm: 4.88 (s, 2H, NH₂), 6.61 (t, 1H, 6.4 Hz), 6.75-6.82 (m, 2H), 6.88-6.94 (m, 2H), 7.02 (d, 2H, CH═CH, 14.5 Hz), 7.10 (d, 1H, 6.3 Hz), 7.57-7.61 (m, 1H), 7.67-7.70 (2H), 8.03-8.09 (m, 2H, NH+ar), 8.19 (d, 1H, 7.7 Hz).

¹³C-NMR (DMSO-d6) δ in ppm: 113.7 (C¹), 115.6 (C³), 115.9 (C^{3′}), 116.8 (C^{6′}), 125.2 (C^{1′}), 126.0 (C^{4′}), 126.5 (C^{5′}), 128.3 (C^{4a}), 129.1 (C⁹), 130.4 (C⁷), 131.7 (2 C, C¹⁰+C¹¹), 132.2 (C⁶), 132.7 (C⁴), 132.8 (C⁸), 134.8 (C^{5a}), 137.2 (C^{9a}), 138.4 (C^{2′}), 144.1 (C^{11a}), 150.7 (C²), 188.7 (C⁵).

GC (method 6) 34.8 min

MS m/z (%): 312 (100, M⁺), 295 (18), 176 (7), 165 (16), 141 (10), 134 (10), 119 (10).

IR (ATR): 1596 (C═O), 1571, 1558, 1497, 1370, 1304, 1257, 1226, 806, 751, 735 cm⁻¹.

### Example 28

### 2-(2-Methoxyphenylamino)-10,11-dihydrodibenzo[a,d]cyclohepten-5-one

1.5 g (5.65 mmol) of 2-acetamidodibenzo[a,d]suberone, 17.0 g (90.9 mmol) of 2-bromoanisole, 0.90 g (6.5 mmol) of K₂CO₃, 2 spatula tips of KI and 2 spatula tips of copper powder are melted at 210° C. (oil bath temperature) and refluxed for 15 h. Cooling is followed by filtration through Fluorisil® and washing with CH₂Cl₂. Purification takes place by column chromatography on SiO₂ with CH₂Cl₂→CH₂Cl₂/EtOH 95+5.

Yield: 0.3 g (16.3%); melting point: 52.5° C.

¹H NMR (DMSO-δ6) δ in ppm: 3.03 (m, 4H, CH₂—CH₂), 3.79 (s, 3H, CH₃), 6.71 (d, 1H), 6.83 (d, 1H), 6.90-7.00 (m, 1H), 7.09 (d, 2H), 7.26-7.35 (m, 3H), 7.43 (d 1H), 7.83 (d, 1H), 7.95 (d, 1H), 8.18 (s, 1H, NH).

¹³C-NMR (DMSO-δ6) δ in ppm: 34.39 (C¹⁰), 36.19 (C¹¹), 55.8 (O—CH₃), 112.43 (C³), 112.85 (C¹), 114.16 (C^{3′}), 120.99 (C^{4′}), 122.69 (C^{6′}), 124.61 (C^{5′}), 126.74 (C⁷), 127.09 (C^{4a}), 128.98 (C⁹), 129.58 (C^{1′}), 130.51 (C⁶), 132.14 (C⁴), 133.55 (C⁸), 139.50 (C^{5a}), 142.02 (C^{9a}), 145.50 (C^{11a}), 149.70 (C²), 152.26 (C^{2′}), 191.02 (C⁵).

MS m/z (%): 329 (100, M⁺), 314 (6, M⁺-CH₃), 296 (5), 196 (23), 183 (24), 165 (6), 151 (6).

IR (ATR): 3399, 3335, 3061, 2937, 2835, 1627, 1579, 1565, 1519, 1494, 1460, 1290, 1268, 1244, 1214, 1110, 1024, 910, 831, 744, 693 cm⁻¹.

C. Preparation of Compounds of the Formula I in which X═O and Y═CH₂

### Example 29

### 8-(2-Aminophenylamino)-6,11-dihydrodibenzo[b,e]oxepin-11-one (45)

### a) 2-Bromomethyl-4-nitrobenzonitrile (39)

3.00 g (18.52 mmol) of 2-methyl-4-nitrobenzonitrile are dissolved in 20 ml of dry tetrachloromethane at about 70° C. Then 3.30 g (18.97 mmol) of N-bromosuccinimide and a little azobisisobutyronitrile and elemental bromine are added. The mixture is refluxed while irradiating with a 500 W spotlight for about 5 h. After the reaction is complete, the succinimide which has deposited on the surface is, after cooling to room temperature, removed by filtration. The filtrate is evaporated, resulting in the product as a yellow-orange oil.

Yield: 3.23 g (50%)

¹H NMR (CDCl₃) δ (ppm): 8.44-8.43 (m, 1H, aryl H), 8.31-8.25 (m, 2H, aryl H), 4.71 (s, 2H, —CH₂Br)

IR (ATR) (cm⁻¹): 1712, 1525, 1348, 1299, 904, 845, 808, 746.

### b) 4-Nitro-2-phenoxymethylbenzonitrile (40)

2.85 g (30.32 mmol) of phenol are dissolved in 10 ml of acetone, and 4.20 g (30.43 mmol) of potassium carbonate and 7.31 g (30.35 mmol) of 2-bromomethyl-4-nitrobenzonitrile are added. The reaction mixture is refluxed at about 70° C. for 5 h and then the solvent is removed. The residue is taken up in ethyl acetate and extracted with 20% strength hydrochloric acid, and the solvent of the organic phase is removed. The resulting dark brown product is purified by RP-18 MPLC column chromatography with acetonitrile/water 6+4, resulting in the yellow product.

Yield: 3.23 g (41.9%); melting point: 112° C.

¹H NMR (CDCl₃) δ (ppm): 8.88 (s, 1H, aryl H), 8.33-8.28 (m, 2H, aryl H), 7.92 (d, 1H, J=8.52 Hz, aryl H), 7.41-7.33 (m, 2H, aryl H), 7.11-6.74 (m, 3H, aryl H), 5.36 (s, 2H, —CH₂—O)

IR (ATR) (cm⁻¹): 1527, 1350, 1244, 1232, 809, 757, 744, 693.

### c) 4-Nitro-2-phenoxymethylbenzoic Acid (41)

0.5 g (1.97 mmol) of 4-nitro-2-phenoxymethylbenzonitrile is dissolved in 25 ml of ethanol at about 80° C. and heated to reflux. Then 7.00 g of KOH are added, and the reaction mixture is again refluxed for about 4 h. Cooling is followed by acidification with 20% strength hydrochloric acid, and the resulting precipitate is filtered off and dried, resulting in the product.

Yield: 0.41 g (76.2%); melting point: 154° C.

IR (ATR) (cm⁻¹): 1691, 1600, 1586, 1497, 1344, 1242, 1274, 1046, 811, 750

### d) 8-Nitro-6H-dibenzo[b,e]oxepin-11-one (42)

0.60 g (2.19 mmol) of 4-nitro-2-phenoxymethylbenzoic acid are dissolved in 10 ml of sulfolane under argon by heating to about 100° C. After the acid has completely dissolved, 15 ml (30.0 g) of polyphosphoric acid are added, and the mixture is stirred at 100° C. for about 2 h. Then ice-water is added, and the precipitated product is filtered off and recrystallized from ethanol.

Yield: 0.54 g (96.6%)

IR (ATR) (cm⁻¹): 1640, 1596, 1524, 1472, 1445, 1299, 1245, 1204, 1138, 1014, 908, 766, 738, 728, 691

### e) 8-Amino-6,11-dihydrodibenzo[b,e]oxepin-11-one (43)

1.00 g (3.92 mmol) of 8-nitro-6H-dibenzo[b,e]oxepin-11-one is dissolved in 10 ml of ethanol, 2.00 g (8.85 mmol) of tin(II) chloride dihydrate are added, and the mixture is stirred at 70° C. for about 2 h. After the mixture has cooled to room temperature, ice-water is added, and sodium hydroxide solution is used to make alkaline. The aqueous phase is extracted with ethyl acetate, and the ethyl acetate phase is evaporated, resulting in the product.

Yield: 0.70 g (79.3%); melting point: 177° C.

¹H NMR (CDCl₃) δ (ppm): 8.35 (d, 1H, J=1.76 Hz, aryl H), 7.92 (d, 1H, J=4.44 Hz, aryl H), 7.44 (t, 1H, J=4.24 Hz, aryl H), 7.14-7.04 (m, 2H, aryl H), 6.68 (d, 1H, J=2.14 Hz, aryl H), 6.53 (s, 1H, aryl H), 5.13 (s, 2H, —CH₂—O—), 4.21 (s, 2H, —NH₂)

IR (ATR) (cm⁻¹): 1602, 1579, 1550, 1303, 1276, 1023, 757, 692.

### f) 8-(2-Nitrophenylamino)-6H-dibenzo[b,e]oxepin-11-one (44)

0.30 g of the product from stage e) is added in small portions to a suspension of 0.10 g (4.17 mmol) of sodium hydride in 10 ml of dimethylformamide. After gas evolution ceases, 0.20 g (1.42 mmol) of 2-fluoronitrobenzene is added, and the mixture is stirred in an ice bath for about 1 h. Ice-water is then added to the reaction mixture, the resulting precipitate is filtered off, and the crude product is recrystallized from methanol.

Yield: 0.30 g (65.1%)

IR (ATR) (cm⁻¹): 1644, 1602, 1571, 1315, 1254, 1145, 739.

### g) 8-(2-Aminophenylamino)-6,11-dihydrodibenzo[b,e]oxepin-11-one (45)

0.35 g (3.92 mmol) of 8-nitro-6H-dibenzo[n.e]oxepin-11-one, 2.00 g (8.85 mmol) of tin(II) chloride dihydrate are reacted by the method described in stage e) using 10 ml of ethanol, resulting in the product.

Yield: 0.16 g (50.0%)

IR (ATR) (cm⁻¹): 1591, 1577, 1546, 1301, 1245, 1208, 767, 736.

### REFERENCES

(1) Kluge, A. F.; Caroon, J. M.; Unger, S. H.; and Ryley, J. F. Tricyclic aryl-substituted anticoccidial azauracils. J. Med. Chem. 1978, 21, 529-536.
(2) Effenberger, F. and Gutmann, R. Electrophilic aromatic substitution. 23. The Fries rearrangement as an equilibrium reaction. Chem. Ber. 1982, 115, 1089-1102.
(3) Zincke, T. and Muller, J. 1,3-Aminophenyl Mercaptan. Chem. Ber. 1913, 46, 775-786.
(4) Ottosen, E. R.; Sørensen, M. D.; Bjoerkling, F.; Skak-Nielsen, T.; Fjording, M. S.; Aaes, H.; and Binderup, L. Synthesis and Structure-Activity Relationship of Aminobenzophenones. A Novel Class of p38 MAP Kinase Inhibitors with High Antiinflammatory Activity. J. Med. Chem. 2003, 46, 5651-5662.
(5) Griffin, R. J.; Meek, M. A.; Schwalbe, C. H.; and Stevens, M. F. G. Structural studies on bioactive compounds. 8. Synthesis, crystal structure and biological properties of a new series of 2,4-diamino-5-aryl-6-ethylpyrimidine dihydrofolate reductase inhibitors with in vivo activity against a methotrexate-resistant tumor cell line. J. Med. Chem. 1989, 32, 2468-2474.
(6) Regan, J.; Breitfelder, S.; Cirillo, P.; Gilmore, T.; Graham, A. G.; Hickey, E.; Klaus, B.; Madwed, J.; Moriak, M.; Moss, N.; Pargellis, C.; Pav, S.; Proto, A.; Swinamer, A.; Tong, L.; and Torcellini, C. Pyrazole Urea-Based Inhibitors of p38 MAP Kinase: From Lead Compound to Clinical Candidate. J. med. Chem. 2002, 45, 2994-3008.
(7) T. Eicher, K. Tiefensee, R. Pick; Synthese von Bryophyten-Inhaltsstoffen.1. Neue Synthesen der Lunularsäure und einiger ihrer Derivate; Synthesis 1988, 7, 525.
(8) A. F. Kluge, J. M. Caroon, S. H Unger; Tricyclic Aryl-Substituted Anticoccidial Azauracils; J. Med. Chem. 1978, 21 (6), 529.
(9) C. G. Collins, S. Pankay, A. Jaklitsch; Serum pretreatment for tricyclic antidepressant drug assays and kit therefor; EP 177344A.
(10) M. Thyes, G. Steiner; Verfahren zur Herstellung von 5H-Dibenzo[a,d]cycloheptan-5-onen; DE 3644462 A.
(11) M. Thyes, G. Steiner; Verfahren zur Herstellung von (Z)-2-(2-Arylethenyl)-arylcarbonsäuren; DE 3644463 A.
(12) F. Effenberger, R. Gutmann; Die Fries-Umlagerung als Gleichgewichtsreaktion; Chem. Ber. 1982, 115 (3), 1089.

1. A dibenzocycloheptane compound of the formula I 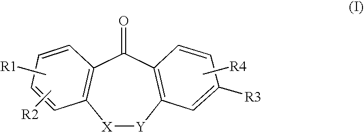
in which
one of the ring atoms X and Y is CH₂ and the other is CH₂, O, S, SO, SO₂ or NR5;
or —X—Y— is —CH₂—CH₂— or —CH═CH—;
R1 is H or C₁-C₆-alkyl;
R2 is H, halogen or C₁-C₄-alkyl-C≡C— which is optionally substituted by an amino group;
R3 is selected from: 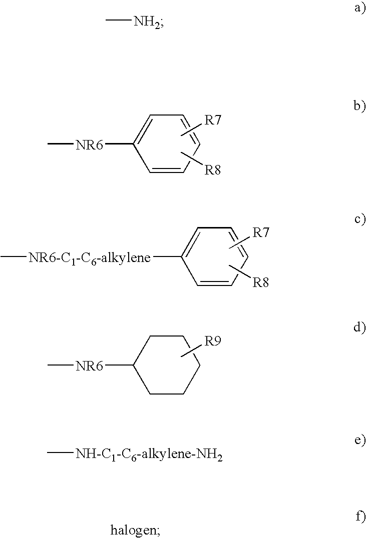
R4 is H, halogen or C₁-C₆-alkyl, or R3 and R4 are bonded to adjacent C atoms of the phenyl ring and form together with these C atoms a 5- or 6-membered aromatic or nonaromatic heterocycle having a nitrogen heteroatom, where the heterocycle may be substituted by one or two C₁-C₆-alkyl groups or may be fused to a cyclohexyl group;
R5 is H or C₁-C₆-alkyl;
R6 is H or C₁-C₆-alkyl;
R7 is selected from:
H,
NH₂,
mono-C₁-C₆-alkylamino,
di-C₁-C₆-alkylamino,
C₁-C₆-alkyl-CONH—,
C₁-C₆-alkyl-NHCONH—,
C₁-C₆-alkyl-O—CO—NH—,
C₁-C₆-alkyl,
C₁-C₆-alkoxy,
NO₂ or
halogen;
R8 is H, NH₂, mono-C₁-C₆-alkylamino, di-C₁-C₆-alkylamino, C₁-C₆-alkoxy, or halogen;
R9 is H or NH₂;
and the physiologically tolerated salts and the solvates of the compounds and of the salts thereof.

2. A compound as claimed in claim 1 of the formula Ia 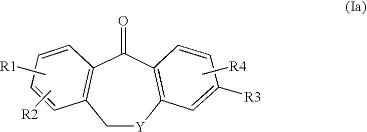
in which Y is O or S, and R1, R2, R3 and R4 have the meanings indicated in claim 1.

3. A compound as claimed in claim 1 of the formula Ib: 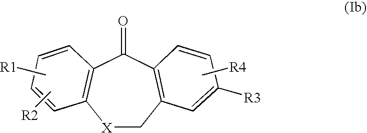
in which X is O or S, and R1, R2, R3 and R4 have the meanings indicated in claim 1.

4. A compound as claimed in claim 1 of the formula Ic: 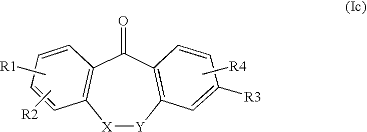
in which —X—Y— is —CH₂—CH₂— or —CH═CH—, and R1, R2, R3 and R4 have the meanings indicated in claim 1.

5. A compound as claimed in claim 1, where R1 and R2 are H.

6. A compound as claimed in claim 1, where R3 is selected from

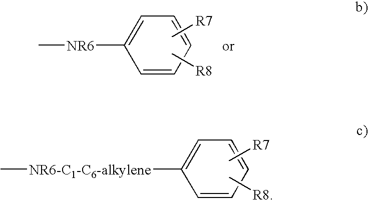

7. A compound as claimed in claim 6, where R3 is selected from

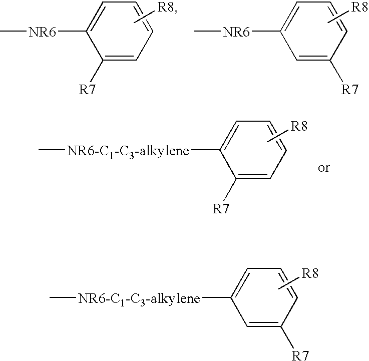

8. A compound as claimed in claim 1, where R4 is H.

9. A compound as claimed in claim 1, where R5 and R6 are H.

10. A compound as claimed in claim 1, where R7 is NH, C₁-C₆-alkyl-CONH—, C₁-C₆-alkyl-NHCONH— or C₁-C₆-alkyl-O—CO—NH—.

11. A compound as claimed in claim 2 of the formula Iaa 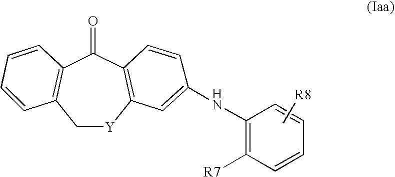
in which Y has the meanings indicated in claim 2, R7 has the meanings indicated in claim 1 or 10, and R8 has the meanings indicated in claim 1.

12. A compound as claimed in claim 4 of the formula Ica: 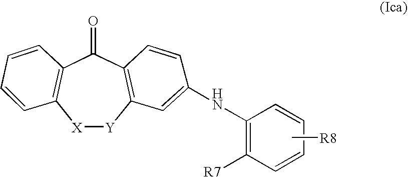
in which
—X—Y— is —CH₂—CH₂— or —CH═CH—, and R7 has the meanings indicated in claim 1 or 10, and R8 has the meaning indicated in claim 1.

13. A compound as claimed in claim 1:
(1) 3-amino-6,11-dihydrodibenzo[b,e]thiepin-11-one
(2) 3-amino-6,11-dihydrodibenzo[b,e]oxepin-11-one
(3) 3-fluoro-6,11-dihydrodibenzo[b,e]oxepin-11-one
(4) 3-(2-aminoanilino)-6,11-dihydrodibenzo[b,e]thiepin-11-one
(5) 3-(2-aminoanilino)-6,11-dihydrodibenzo[b,e]oxepin-11-one
(6) 3-(4-aminoanilino)-6,11-dihydrodibenzo[b,e]oxepin-11-one
(7) 3-(2-fluoro-4-aminoanilino)-6,11-dihydrodibenzo[b,e]oxepin-11-one
(8) 2,11,12,13,14-pentahydro-10H-(benzo[e]oxepin)[2,3-c]carbazol-7-one
(9) 2-hydro-11,12-dimethyl-10H-(benzo[e]oxepin)[2,3-e]indol-7-one
(10) 3-(3-aminoanilino)-6,11-dihydrodibenzo[b,e]oxepin-11-one
(11) 3-(2,4-diaminophenylamino)-6,11-dihydrodibenzo[b,e]oxepin-11-one
(12) 3-(2-aminobenzylamino)-6,11-dihydrodibenzo[b,e]oxepin-11-one
(13) 3-(2-aminoethylamino)-6,11-dihydrodibenzo[b,e]oxepin-11-one
(14) 3-(cis-2-amino-cyclohexylamino)-6,11-dihydrodibenzo[b,e]oxepin-11-one
(15) 3-((1R)-trans-2-aminocyclohexylamino)-6,11-dihydrodibenzo[b,e]oxepin-11-one
(16) 3-((1S)-trans-2-aminocyclohexylamino)-6,11-dihydrodibenzo[b,e]oxepin-11-one
(17) 3-(2-acetamidoanilino)-6,11-dihydrodibenzo[b,e]oxepin-11-one
(18) 1-ethyl-3-[2-(11-oxo-6,11-dihydrodibenzo[b,e]oxepin-3-ylamino)phenyl]urea
(19) ethyl [2-(11-oxo-6,11-dihydrodibenzo[b,e]oxepin-3-ylamino)phenyl]carbamalate
(20) 3-(2-methylaminophenylamino)-6,11-dihydrodibenzo[b,e]oxepin-11-one
(21) 3-(2,4-difluorophenylamino)-6,11-dihydrodibenzo[b,e]oxepin-11-one
(22) 3-(2,4-dichlorophenylamino)-6,11-dihydrodibenzo[b,e]oxepin-11-one
(23) 3-(2,4-dibromophenylamino)-6,11-dihydrodibenzo[b,e]oxepin-11-one
(24) 2-(2-aminophenylamino)-10,11-dihydrodibenzo[a,d]cycloheptan-5-one
(25) 2-(4-aminophenylamino)-10,11-dihydrodibenzo[a,d]cycloheptan-5-one
(26) 2-(4-aminophenylamino)dibenzo[a,d]cyclohepten-5-one
(27) 3-acetamido-6,11-dihydrodibenzo[b,e]thiepin-11-one
(28) 3-(4-fluoro-2-nitrophenylamino)-6,11-dihydrodibenzo[b,e]oxepin-11-one
(29) 8-(2-aminophenyamino)-6,11-dihydrodibenzo[b,e]oxepin-11-one
(30) 2-(2-methoxyphenylamino)-10,11-dihydrodibenzo[a,d]cyclohepten-5-one.

14. A pharmaceutical composition comprising at least one compound of the formula I as claimed in claim 1, where appropriate together with physiologically tolerated excipients.

15. A method of producing a pharmaceutical composition, comprising introducing into a pharmaceutical carrier at least one compound of the formula I as claimed in claim 1 for producing a pharmaceutical composition for at least one of immunomodulation or for inhibiting the release of at least one of IL-1β or TNF-α.

16. A method of treatment comprising administering to a mammal at least one compound of the formula I as claimed in claim 1 for the treatment of autoimmune diseases, cancer, rheumatoid arthritis, gout, septic shock, osteoporosis, neuropathic pain, HIV dissemination, HIV dementia, viral myocarditis, insulin-dependent diabetes, periodontal disorders, restenosis, alopecia, T-cell depletion in HIV infections or AIDS, psoriasis, acute pancreatitis, rejection reactions with allogeneic transplants, allergy-related inflammation of the lungs, arteriosclerosis, multiple sclerosis, cachexia, Alzheimer's disease, stroke, icterus, ulcerative colitis, Crohn's disease, inflammatory bowel disease (IBD), ischemia, congestive heart failure, pulmonary fibrosis, hepatitis, glioblastoma, Guillain-Barré syndrome, systemic lupus erythematosus, adult respiratory distress syndrome (ARDS) or respiratory distress syndrome.

17. A method for at least one of immunomodulation or regulating at least one of release of IL-1β or TNF-α in a person requiring such a treatment, where an amount, which at least one of has an immunomodulating effect or regulates or inhibits the release of at least one of IL-1β or TNF-α, of a compound of the formula I as claimed in claim 1 is administered to the person.
